(19) United States
(12) Patent Application Publication
(10) Pub. No.: US 2023/0420075 A1
(43) Pub. Date: Dec. 28, 2023

(54) ACCELERATORS FOR A GENOTYPE IMPUTATION MODEL

(71) Applicant: Illumina Software, Inc., San Diego, CA (US)

(72) Inventors: Mark David Hahm, Hartland, WI (US); Sven Bilke, Bethesda, MD (US); Andrew Christopher Du Preez, Poway, CA (US); Michael Ruehle, Fort Worth, TX (US)

(21) Appl. No.: 18/342,580

(22) Filed: Jun. 27, 2023

Related U.S. Application Data
(60) Provisional application No. 63/367,105, filed on Jun. 27, 2022.

(51) Int. Cl. G16B 20/20; G16B 40/00

(52) U.S. Cl. CPC G16B 20/20; G16B 40/00

(57) ABSTRACT

This disclosure describes methods, non-transitory computer readable media, and systems that can determine allele likelihoods of a genomic region exhibiting certain haplotype alleles using one or both of consolidated computations and data exchanges across specialized hardware. For instance, the disclosed systems can determine an intermediate allele likelihood of a genomic region comprising a haplotype allele by running a single-pass-concurrent-multiplication operation. In some cases, the disclosed systems determine and store subsets of intermediate allele likelihoods corresponding to marker-variant groups and extemporaneously generate sets of intermediate allele likelihoods for a set of marker variants by using the intermediate-allele-likelihood subsets as hot-start points. In further embodiments, the disclosed systems determine running sums of intermediate allele likelihoods of a genomic region exhibiting haplotype alleles for haplotypes given one marker variant and use the running sums as inputs to determine intermediate allele likelihoods of the genomic region exhibiting the haplotype alleles given another marker variant.

### CROSS-REFERENCE TO RELATED APPLICATIONS

The present application claims the benefit of, and priority to, U.S. Provisional Application No. 63/367,105, entitled “ACCELERATORS FOR A GENOTYPE IMPUTATION MODEL,” filed on Jun. 27, 2022. The aforementioned application is hereby incorporated by reference in its entirety.

### BACKGROUND

In recent years, biotechnology firms and research institutions have improved hardware and software platforms for sequencing nucleotides and determining nucleobase calls for genomic samples. For instance, some existing sequencing machines and sequencing-data-analysis software (together “existing sequencing systems”) determine individual nucleobases within sequences by using conventional Sanger sequencing or by using sequencing-by-synthesis (SBS) methods. When using SBS, existing sequencing systems can monitor many thousands of oligonucleotides being synthesized in parallel from templates to predict nucleobase calls for growing nucleotide reads. For instance, a camera in many existing sequencing systems captures images of irradiated fluorescent tags incorporated into oligonucleotides. After capturing such images, some existing sequencing systems process the image data from the camera and determine nucleobase calls for nucleotide reads corresponding to the oligonucleotides. Based on a comparison of the nucleobase calls for such reads and a reference genome, existing systems utilize a variant caller to identify variants in a genomic sample, such as single nucleotide polymorphisms (SNPs), insertions or deletions (indels), or other variants within the genomic sample.

Despite these recent advances, existing sequencing systems sometimes inaccurately determine base calls or collect an insufficient number of (or seemingly contradictory) nucleotide reads, especially for nucleobases in low-read-coverage genomic regions. For certain genomic regions of a genomic sample, existing sequencing systems often use a genotype imputation model to impute nucleobase calls and phase haplotypes based on detected variants in the genomic sample. For instance, existing sequencing systems frequently use various types of hidden Markov models (HMM) customized for imputing genotypes to impute nucleobase calls for certain genomic regions, such as by using Genotype Likelihoods Imputation and PhaSing mEthod (GLIMPSE) or IMPUTE. While such HMMs have improved the accuracy of imputing genotypes, existing sequencing systems that employ genotype imputation models frequently consume significant computer processing, require significant memory to store data generated by the genotype imputation model, and execute the genotype imputation model with inefficient latencies of downtime for processors.

As just suggested, existing sequencing systems consume inordinate computer processing and time when executing an HMM for genotype imputation. For example, some existing sequencing systems running a single thread on a central processing unit (CPU) consume an average of around 17.5 hours to phase and impute haplotype allele likelihoods for a single marker allele corresponding to a genomic region. Approximately 80% of such phasing and imputation computation comes from both the HMM computation and Burrows-Wheeler transforms (BWT), where the HMM computation consumes roughly 60% and BWT computation consumes roughly 20% of the computation time. While BWT computation can be amortized and diminished significantly as a percentage across large batches of genomic samples, the HMM computation time on a single CPU thread can still consume roughly 10 or more hours (e.g., 600 to 640 minutes).

In addition to consuming significant time and computer processing, existing sequencing systems can consume significant memory when executing an HMM for genotype imputation. For instance, in some cases, existing sequencing systems determine and save values for haplotype allele likelihoods in a haplotype matrix corresponding to 50 million cells for a collection of marker variants and haplotypes from a haplotype reference panel. Given 50 million cells for a single haplotype matrix, existing sequencing systems that determine 40,000 haplotype calls based on 40,000 haplotype matrices in a time period must determine values corresponding to 2 trillion cells. Because some HMMs for genotype imputation, such as GLIMPSE, require existing sequencing systems to determine values once for an alpha pass—and twice for a beta pass of the HMM—for each haplotype matrix, existing sequencing systems can determine and save values in across many haplotype matrices of around 6 trillion cells in total for to compute an HMM-based genotype imputation for multiple genomic regions. While hardware on sequencing devices and servers have increased memory, chips for a Field Programmable Gate Array (FPGA) or other configurable processors often include around 32 or 64 gigabytes of memory on the chip, which is barely sufficient or insufficient memory to store data for a single haplotype matrix.

Beyond taxing processing time and memory, some existing sequencing systems inefficiently perform an HMM for genotype imputation with latency periods for a processor. For instance, in some cases, some existing sequencing systems determine a sum of all intermediate allele likelihoods for one marker variant and various haplotypes from a haplotype reference panel—based on both alpha pass and beta pass values—before even determining individual intermediate allele likelihoods for a subsequent marker variant. Because all intermediate allele likelihoods must be summed and allele likelihoods determined for one marker variant before determining individual intermediate allele likelihoods for another marker variant, the processor used by existing sequencing systems often waits through a latency period for one or both of summing adjacent-marker intermediate allele likelihoods and generating allele likelihoods. For an HMM-based genotype imputation that can require a haplotype matrix of 50 million cells—and 40,000 individual haplotype matrices—such computational latency is inefficient and wastes time that the processor could otherwise use to further compute allele likelihoods.

As the memory size and computation time described above suggests, an existing sequencing system would require significant throughput of input and output values to efficiently execute an HMM-based genotype imputation. Because such imputation can require storing or transferring large amounts of data—such as certain input values for a haplotype matrix with millions, billions, or trillions of cells—existing HMM-based genotype imputations further tax the bandwidth of high-speed buses, such as a Peripheral Component Interconnect Express (PCIe), or other interfaces that connect processor cards with other hardware within a computing device. Any bottleneck on PCIe throughput or other interface throughput can significantly slow HMM-based genotype imputations.

These, along with additional problems and issues exist in existing sequencing systems.

### SUMMARY

This disclosure describes one or more embodiments of systems, methods, and non-transitory computer readable storage media that solve one or more of the problems described above or provide other advantages over the art. To expedite computer processing or efficiently redistribute the memory load of a genotype imputation model, the disclosed system can determine allele likelihoods of a genomic region exhibiting certain haplotype alleles using consolidated computations, efficient data transfers, or customized architecture. For instance, the disclosed systems can determine an intermediate allele likelihood of a genomic region comprising a haplotype allele—given a marker variant and a reference panel haplotype—by running a single, pass-concurrent multiplication operation on a processor. In some cases, the disclosed systems determine and store subsets of intermediate allele likelihoods corresponding to marker-variant groups and generate full sets of intermediate allele likelihoods for a set of marker variants by using the intermediate-allele-likelihood subsets as hot-start points. In further embodiments, the disclosed systems determine running sums of intermediate allele likelihoods of a genomic region exhibiting haplotype alleles for one or more haplotypes given one marker variant and using the running sums as running inputs to determine individual intermediate allele likelihoods of the genomic region exhibiting the haplotype alleles given another marker variant—without latency periods for summing intermediate allele likelihoods and/or generating allele likelihoods.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which an accelerated genotype-imputation system can operate in accordance with one or more embodiments of the present disclosure.

FIGS. 2A-2B illustrate the accelerated genotype-imputation system utilizing a haplotype matrix to perform a hidden Markov model (HMM)-based genotype imputation model to determine posterior genotype likelihoods for a genomic region of multiple genomic samples in accordance with one or more embodiments of the present disclosure.

FIGS. 3A-3B illustrate the accelerated genotype-imputation system determining an intermediate allele likelihood of a genomic region comprising a haplotype allele by running consolidated operations on a processor in accordance with one or more embodiments of the present disclosure.

FIGS. 4A-4B illustrate the accelerated genotype-imputation system determining and storing intermediate-allele-likelihood subsets as hot-start points and generating sets of intermediate allele likelihoods for a set of marker variants by using the intermediate-allele-likelihood subsets in accordance with one or more embodiments of the present disclosure.

FIGS. 5A-5B illustrate the accelerated genotype-imputation system determining running sums of intermediate allele likelihoods of a genomic region exhibiting haplotype alleles for one or more haplotypes given one marker variant and using the running sums as running inputs to determine individual intermediate allele likelihoods of the genomic region exhibiting the haplotype alleles given another marker variant in accordance with one or more embodiments of the present disclosure.

FIG. 6 illustrates the accelerated genotype-imputation system storing haplotype-allele-indicator data for a haplotype matrix on a memory device and accessing the stored haplotype-allele indicator data to determine values as part of a pass across a haplotype matrix in accordance with one or more embodiments of the present disclosure.

FIG. 7 illustrates an accelerated computation engine of the accelerated genotype-imputation system in accordance with one or more embodiments of the present disclosure.

FIG. 8 illustrates a data flow engine of the accelerated genotype-imputation system orchestrating data inputs and outputs of a cluster of accelerated computation engines and an on-board memory device of a configurable processor board in accordance with one or more embodiments of the present disclosure.

FIG. 9 illustrates a schematic diagram of an accelerated computation engine comprising a core, surrounding interfaces, and other hardware in accordance with one or more embodiments of the present disclosure.

FIG. 10 illustrate series of acts for determining an intermediate allele likelihood of a genomic region comprising a haplotype allele by running consolidated operations on a processor in accordance with one or more embodiments of the present disclosure.

FIG. 11 illustrate series of acts for determining and storing intermediate-allele-likelihood subsets as hot-start points corresponding to marker-variant groups and extemporaneously generating sets of intermediate allele likelihoods for a set of marker variants by using the intermediate-allele-likelihood subsets in accordance with one or more embodiments of the present disclosure.

FIG. 12 illustrate series of acts for determining running sums of intermediate allele likelihoods of a genomic region exhibiting haplotype alleles for one or more haplotypes given one marker variant and using the running sums as running inputs to determine individual intermediate allele likelihoods of the genomic region exhibiting the haplotype alleles given another marker variant in accordance with one or more embodiments of the present disclosure.

FIG. 13 illustrates a block diagram of an example computing device in accordance with one or more embodiments of the present disclosure.

### DETAILED DESCRIPTION

This disclosure describes one or more embodiments of an accelerated genotype-imputation system that can determine intermediate allele likelihoods of a genomic region exhibiting certain haplotype alleles as part of a genotype imputation model by using consolidated computations or efficient data transfers across specialized hardware. For instance, the accelerated genotype-imputation system can determine an intermediate allele likelihood of a genomic region comprising a haplotype allele—given a particular marker variant and a haplotype from a haplotype reference panel—by running a single, pass-concurrent multiplication operation on a processor, rather than multiple pass-concurrent multiplication operations. In some cases, the accelerated genotype-imputation system (i) determines and stores subsets of intermediate allele likelihoods corresponding to groups of marker variants and (ii) generates sets of intermediate allele likelihoods by using the intermediate-allele-likelihood subsets as hot-start points for a full pass of intermediate allele likelihoods. The accelerated genotype-imputation system can perform (i) and (ii) without storing multiple full sets of intermediate allele likelihoods on a processor chip during real-time processing. In certain further embodiments, the accelerated genotype-imputation system determines running sums of intermediate allele likelihoods of a genomic region exhibiting haplotype alleles for one or more haplotypes given one marker variant and using the running sums as running inputs to determine intermediate allele likelihoods of the genomic region exhibiting the haplotype alleles given another marker variant. By using such running sums, the accelerated genotype-imputation system avoids idle latency periods of a processor summing adjacent-marker intermediate allele likelihoods and/or generating allele likelihoods that slowdown existing sequencing systems.

As suggested above, the accelerated genotype-imputation system applies a genotype imputation model, such as a hidden Markov model (HMM)-based model, to nucleotide reads from a genomic region of a genomic sample to determine posterior genotype likelihoods and haplotype calls for the genomic region. To illustrate, in some embodiments, the accelerated genotype-imputation system determines prior genotype likelihoods that a genomic region comprises a particular genotype (e.g., a reference allele or alternate allele), where the genomic region corresponds to variable positions or coordinates of a haplotype reference panel. Such prior genotype likelihoods are based on nucleotide reads from the genomic sample and quality scores for the nucleotide reads. The accelerated genotype-imputation system further deconvolves a vector of the prior genotype likelihoods to two independent vectors of haplotype allele likelihoods (or, simply, haplotype likelihoods), where each vector corresponds to one of two complementary haplotypes. Based on the haplotype likelihoods from the independent vectors, the accelerated genotype-imputation system imputes two target haplotypes as haplotype calls using a haploid version of an HMM. The accelerated genotype-imputation system further determines (and updates) the phase of the two imputed haplotypes. In some embodiments, for instance, the accelerated genotype-imputation system uses Genotype Likelihoods Imputation and PhaSing mEthod (GLIMPSE) as a genotype imputation model, as described by Simone Rubinacci et al., “Efficient Phasing and Imputation of Low-coverage Sequencing Data Using Large Reference Panels,” 53 Nature Genetics 120-126 (2021) (hereinafter, Rubinacci), which is hereby incorporated by reference in its entirety.

The disclosed accelerated genotype-imputation system introduces and utilizes consolidated computations or unique architecture to efficiently determine intermediate allele likelihoods of a genomic region exhibiting certain haplotype alleles as part of GLIMPSE or another genotype imputation model. The follow paragraphs briefly introduce various embodiments of the accelerated genotype-imputation system.

A. Single, Pass-Concurrent Operations

As suggested above, the accelerated genotype-imputation system determines an intermediate allele likelihood of a genomic region comprising a haplotype allele by running a single, pass-concurrent multiplication operation for a given marker variant and haplotype. To perform such an operation, in some embodiments, the accelerated genotype-imputation system identifies a haplotype reference panel for a genomic region of a genomic sample as part of a genotype imputation model. The accelerated genotype-imputation system further accesses a first transition-aware allele-likelihood factor (e.g., Q[m][Allele]*P1[m]) corresponding to a haplotype allele from the haplotype reference panel and a second transition-aware allele-likelihood factor (e.g., Q[m][Allele]*P0[m]) corresponding to the haplotype allele. By combining the first allele-likelihood factor and an adjacent-marker intermediate allele likelihood (e.g., A′[m−1][k]) of the genomic region comprising the haplotype allele given an adjacent marker variant, the accelerated genotype-imputation system can perform a single, pass-concurrent multiplication operation and generate an adjacent-marker-transition-factor-aware allele likelihood (e.g., Q[m][Allele]*P1[m]*A′[m−1]) for the marker variant and a haplotype. Based on the adjacent-marker-transition-factor-aware allele likelihood and the second transition-aware allele-likelihood factor, the accelerated genotype-imputation system further determines, for the given marker variant and the haplotype, an intermediate allele likelihood of the genomic region comprising the haplotype allele.

By performing such a single, pass-concurrent multiplication operation, the accelerated genotype-imputation system expedites the computer processing time to determine intermediate allele likelihoods and output allele likelihoods over the slower computer processing time of existing sequencing systems. As noted above, some existing sequencing systems running a single thread on a central processing unit (CPU) consume an average of around 17.5 hours to phase and impute haplotype allele likelihoods for a single marker allele corresponding to a genomic region—where the HMM computation time on the single CPU thread can consume roughly 10 hours. As explained further below, the hours-long computer processing time comes in part from a sequencing system performing 3 multiplication operations to determine intermediate allele likelihoods for each given pair of a marker variant and a haplotype and 3,000 multiplication operations for each haplotype of a haplotype reference panel (e.g., organized in a row).

In contrast to existing sequencing systems, in some embodiments, the disclosed accelerated genotype-imputation system performs a single, pass-concurrent multiplication operation to determine intermediate allele likelihoods for each given pair of a marker variant and a haplotype and roughly 1,000 multiplication operations for each haplotype of a haplotype reference panel (e.g., organized in a row) due to such consolidated multiplication operations. Together with other consolidated operations or other embodiments, the accelerated genotype-imputation system can reduce computer processing time of a single processor thread to perform approximately 40,000 HMM-computation tasks from roughly 10 or more hours (e.g., 600-640 minutes) to approximately 60 seconds, thereby expediting processing time by 600 times.

B. Hot-Start Intermediate-Allele-Likelihood Subsets

As further noted above, in some cases, the accelerated genotype-imputation system determines and store subsets of intermediate allele likelihoods corresponding to marker-variant groups and extemporaneously generate sets of intermediate allele likelihoods by using the subsets of intermediate allele likelihoods as hot-start points for determining a full pass of intermediate allele likelihoods. To determine and utilize such hot-start likelihoods, in some embodiments, the accelerated genotype-imputation system determines first-pass intermediate allele likelihoods of a genomic region from a genomic sample comprising haplotype alleles corresponding to a set of haplotypes given a set of maker variants. The accelerated genotype-imputation system further stores, on dynamic random-access memory (DRAM) or other memory device, a subset of first-pass intermediate allele likelihoods corresponding to a subset of marker variants for groups of marker variants. The accelerated genotype-imputation system subsequently uses the stored subset of first-pass intermediate allele likelihoods to initialize allele-likelihood determinations at the groups of marker variants—thereby regenerating the first-pass intermediate allele likelihoods. The accelerated genotype-imputation system also uses the stored subset of first-pass intermediate allele likelihoods to determine second-pass intermediate allele likelihoods of the genomic region comprising the haplotype alleles corresponding to the set of marker variants and the set of haplotypes. Based on the regenerated first-pass intermediate allele likelihoods and the second-pass intermediate allele likelihoods, the accelerated genotype-imputation system generates allele likelihoods of the genomic region comprising the haplotype alleles.

By determining and using such intermediate-allele-likelihood subsets as hot-start points, the accelerated genotype-imputation system intelligently and efficiently redistributes data between memory devices, reduces data storage, and increase on-chip bandwidth. As noted above, some existing sequencing systems employing HMMs for genotype imputation, such as GLIMPSE, determine and save values in a haplotype matrix of around 50 million cells. The data for such a haplotype matrix would saturate or prove too much to store on the on-chip memory for a Field Programmable Gate Array (FPGA) or other processors of existing sequencing systems. To reduce and redistribute the data of such an enormous haplotype matrix, in some embodiments, the accelerated genotype-imputation system determines and store intermediate-allele-likelihood subsets corresponding to marker-variant groups and uses intermediate-allele-likelihood subsets as hot-start points for determining a full pass of intermediate allele likelihoods.

By determining and storing intermediate-allele-likelihood subsets, the accelerated genotype-imputation system can exponentially reduce and transfer data dependent on a size of marker-variant groups or windows. In some embodiments, for instance, the accelerated genotype-imputation system reduces memory load by 100 times by determining and storing intermediate-allele-likelihood subsets corresponding to each marker variant from a 100-count marker-variant group or reduces memory load by 1,000 times by determining and storing intermediate-allele-likelihood subsets corresponding to each marker variant from a 1,000-count marker-variant group. As further explained below, in some embodiments, the size of the marker-variant group controls the exponent by which the accelerated genotype-imputation system reduces memory load and data transfer.

C. Running Sums of Adjacent-Marker Intermediate Allele Likelihoods

In addition to pass-concurrent multiplication operations and hot-start intermediate-allele-likelihood subsets, the accelerated genotype-imputation system can determine running sums of intermediate allele likelihoods of a genomic region exhibiting haplotype alleles for one or more haplotypes given one marker variant and using the running sums as running inputs to determine individual intermediate allele likelihoods of the genomic region exhibiting the haplotype alleles given another marker variant. To leverage such running sums, in some embodiments, the accelerated genotype-imputation system identifies a haplotype reference panel for a genomic region of a genomic sample as part of a genotype imputation model. The accelerated genotype-imputation system further (i) determines, for an adjacent marker variant, a running sum of a first subset of intermediate allele likelihoods of the genomic region comprising a first type of haplotype allele from one or more haplotypes of the haplotype reference panel and (ii) determines, for the adjacent marker variant, a running sum of a second subset of intermediate adjacent-allele likelihoods of the genomic region comprising a second type of haplotype allele from the one or more haplotypes. Based on the running sums, the accelerated genotype-imputation system determines, for a marker variant, sums of intermediate allele likelihoods of the genomic region comprising haplotype alleles from haplotypes of the haplotype reference panel.

By determining and using such running sums of intermediate allele likelihoods, the accelerated genotype-imputation system removes or reduces latency periods for one or both of summing adjacent-marker intermediate allele likelihoods and generating allele likelihoods. As noted above, some existing sequencing systems sum intermediate allele likelihoods and determine allele likelihoods for one marker variant before determining individual intermediate allele likelihoods for another marker variant, thereby causing the processor of an existing sequencing systems to wait through a latency period for one or both of summing adjacent-marker intermediate allele likelihoods and generating allele likelihoods for the other marker variant.

In contrast to existing sequencing systems, in some embodiments, the accelerated genotype-imputation system determines running sums of intermediate allele likelihoods of a genomic region exhibiting haplotype alleles for one or more haplotypes given one marker variant as running inputs—and without a conventional latency—for determining individual intermediate allele likelihoods of the genomic region exhibiting the haplotype alleles given another marker variant. Without such latency periods summing adjacent-marker intermediate allele likelihoods and generating allele likelihoods, the accelerated genotype-imputation system expedites determining haplotype allele likelihoods for a genotype imputation model faster than existing sequencing systems. Together with other consolidated operations or other embodiments, the accelerated genotype-imputation system can reduce computer processing time of a single processor thread to perform approximately 40,000 HMM-computation tasks from roughly 10 or more hours (e.g., 600-640 minutes) to approximately 60 seconds, thereby expediting processing time by 600 times.

D. Customized Hardware Architecture

To facilitate one or more of the consolidated computations or data storage, in some embodiments, the accelerated genotype-imputation system utilizes a customized architecture. For example, the accelerated genotype-imputation system can store intermediate-allele-likelihood subsets on (and access from) dynamic random-access memory (DRAM) or another memory device to hot-start determining a full pass of intermediate allele likelihoods. As a further example, the accelerated genotype-imputation system can use a data flow engine as part of a configurable processor to (i) que and manage HMM-computation tasks for a corresponding cluster of accelerated computation engines and (ii) distribute input values to individual accelerated computation engines from a cluster for determining intermediate allele likelihoods (or other HMM-computation tasks) for columns or a matrix. In some cases, for instance, the accelerated genotype-imputation system sends, from a data flow engine to respective accelerated computation engines, respective sets of input values (e.g., allele-likelihood factors, transition coefficients, and haplotype-allele values) and uses the respective accelerated computation engines to determine respective sets of intermediate allele likelihoods corresponding to respective subsets of marker variants and respective subsets of haplotypes—based on the respective sets of input values.

As suggested above, the disclosed accelerated genotype-imputation system uses a customized architecture that facilitates faster throughput of input and output values for allele likelihoods in a genotype imputation model than conventional and undifferentiated architectures of existing sequencing systems. For example, the accelerated genotype-imputation system can use an off-chip DRAM or other memory device to store and quickly transfer intermediate-allele-likelihood subsets for hot-starting, rather than slowing throughput down by relying on on-chip memory to store values for intermediate allele likelihoods. As noted above, existing HMM-based genotype imputations tax the bandwidth of high-speed buses, such as a Peripheral Component Interconnect Express (PCIe), or other interfaces with values for a haplotype matrix of 50 million cells—sometimes going through 40,000 such matrices. By storing on and accessing from on-chip DRAM (or other on-chip memory) haplotype-allele-indicator data for a haplotype matrix, in some embodiments, the accelerated genotype-imputation system can generate allele likelihoods utilizing a hidden Markov haploid genotype imputation model or a hidden Markov diploid genotype imputation model with 4 or more gigabytes of PCIe bandwidth than existing sequencing systems.

By using a data flow engine as part of a configurable processor and orchestrating data flow to clusters of accelerated computation engines, in some embodiments, the accelerated genotype-imputation system avoids latency periods and determine allele likelihoods for different haplotypes and marker variants in parallel. Indeed, as explained further below, the data flow engine of the disclosed accelerated genotype-imputation system can efficiently distribute input and output values to different clusters of accelerated computation engines to determine allele likelihoods from the equivalent of 6 trillion cells across multiple haplotype matrices in approximately 60 seconds.

As illustrated by the foregoing discussion, the present disclosure utilizes a variety of terms to describe features and advantages of the accelerated genotype-imputation system. As used herein, the term “genomic sample” refers to a target genome or portion of a genome undergoing sequencing. For example, a sample genome includes a sequence of nucleotides isolated or extracted from a sample organism (or a copy of such an isolated or extracted sequence). In particular, a sample genome includes a full genome that is isolated or extracted (in whole or in part) from a sample organism and composed of nitrogenous heterocyclic bases. A sample genome can include a segment of deoxyribonucleic acid (DNA), ribonucleic acid (RNA), or other polymeric forms of nucleic acids or chimeric or hybrid forms of nucleic acids noted below. In some cases, the sample genome is found in a sample prepared or isolated by a kit and received by a sequencing device.

As also used herein, the term “haplotype” refers to nucleotide sequences that are present in an organism (or present in organisms from a population) and inherited from one or more ancestors. In particular, a haplotype can include alleles (or other nucleotide sequences) present in organisms of a population and inherited together by such organisms respectively from a single parent. In one or more embodiments, haplotypes include a set of SNPs on the same chromosome that tend to be inherited together. As described below, in some cases, a haplotype from a haplotype reference panel can be represented as “k,” and a row of different haplotypes from the haplotype reference panel can be represented as “K.” Additionally, an “imputed haplotype” refers to a haplotype that is estimated or statistically inferred to be present in a sample genome. For instance, an imputed haplotype can be a statistically inferred haplotype for a genomic coordinate or region based on SNPs surrounding or flanking the genomic coordinate or region. As indicated above, an imputed haplotype can include SNPs or other variant-nucleotide-base calls that surround a target genomic region and that upon which the customized sequencing system imputes the haplotype.

Relatedly, the term “haplotype allele” refers to a version of a nucleobase or nucleotide sequence at a genomic coordinate or genomic region corresponding to a haplotype, such as a haplotype for a genomic region encoding for a gene or a non-coding region. In particular, a haplotype allele includes one of two or more versions of a nucleobase or a nucleotide sequence at a genomic coordinate or region that tend to be inherited together in combination as part of a haplotype. As part of a haplotype, in some cases, a combination of haplotype alleles may be inherited by an organism as part of a single gene or across multiple genes. In some cases, this disclosure describes different types of haplotype alleles. For instance, in some embodiments, one type of haplotype allele may refer to a sample reference haplotype allele, and another type of haplotype allele may refer to a sample alternate haplotype allele. While this disclosure sometimes describes a first type and a second type of haplotype alleles corresponding to a particular haplotype, in some embodiments, a haplotype may include more than two types of haplotype alleles (e.g., a sample reference haplotype allele and multiple sample alternate haplotype alleles).

In some cases, a haplotype or its constituent haplotype alleles are represented by a haplotype reference panel. As used herein, a “haplotype reference panel” refers to a digital collection or database of haplotypes from genomic samples for which one or more ancestral or progenitorial haplotypes have been determined. In some cases, a haplotype reference panel includes a digital database of haplotypes from genomic samples representative of (or common among) an organism's population and for which multiple ancestral or progenitorial haplotypes have been determined. In some cases, the accelerated genotype-imputation system uses a haplotype reference panel developed by the Haplotype Reference Consortium (HRM), 1000 Genomes Project, or Illumina, Inc.

Relatedly, the term “genotype imputation model” refers to an algorithm or model for imputing genotypes of genomic regions based on sequencing data from a genomic sample and haplotypes corresponding to respective genomic regions. In particular, a genotype imputation model includes a hidden Markov model (HMM)-based algorithm or model for imputing genotypes of genomic regions and phasing haplotypes based on sequencing data from a genomic sample and haplotypes corresponding to respective genomic regions from a haplotype reference panel. As indicated above, in some cases, a genotype imputation model includes GLIMPSE. Alternatively, a genotype imputation models includes fastPHASE, BEAGLE, MACH, or IMPUTE.

As part of imputing genotype, in some cases, the accelerated genotype-imputation system determines allele likelihoods. As used herein, the term “allele likelihood” refers to a likelihood that a genomic region exhibits or comprises a haplotype allele corresponding to a haplotype. For instance, in some embodiments, an allele likelihood includes a statistical likelihood that a genomic region of a genomic sample exhibits or comprises a sample reference haplotype allele or a sample alternate haplotype allele for a particular haplotype from a haplotype of a haplotype reference panel. As described below, in some cases, an allele likelihood can be represented as (i) R0 for a likelihood that a genomic region of a genomic sample comprises a sample reference haplotype allele of a particular haplotype or (ii) R1 for a likelihood that a genomic region of a genomic sample comprises a sample alternate haplotype allele of a particular haplotype. Accordingly, in some cases, an allele likelihood represents a posterior genotype likelihood generated by a genotype imputation model.

Relatedly, the term “intermediate allele likelihood” refers to value representing a provisional or preliminary likelihood that a genomic region exhibits or comprises a haplotype allele corresponding to a haplotype. For instance, in some embodiments, an intermediate allele likelihood includes a value representing a provisional or preliminary likelihood that a genomic region of a genomic sample exhibits or comprises a sample reference haplotype allele or a sample alternate haplotype allele for a particular haplotype from a haplotype of a haplotype reference panel given a target marker variant. As described further below, in some cases, an intermediate allele likelihood can be represented as A[m][k] and called alpha values or, alternatively, represented as B[m][k] and called beta values. While this disclosure primarily uses A[m][k] as example notation for an intermediate allele likelihood in an alpha pass, the notation B[m][k] may be used interchangeably for an intermediate allele likelihood in a beta pass.

Relatedly, the term “marker variant” refers to a variant at a polymorphic site in a population. In particular, a marker variant includes one of two or more alleles present among a population at a polymorphic genomic coordinate or genomic region at a frequency greater than a threshold frequency, such as greater than 1% of a population. In some cases, a marker variant includes SNPs present at a polymorphic genomic coordinate among a human population. Additionally, or alternatively, a marker variant can include insertions or deletions (indels), structural variants, or other variants at polymorphic sites among a population. As described further below, in some cases, a marker variant or a target marker variant is represented as m or [m]. By contrast, the term “adjacent marker variant” refers to a marker variant that is ordered before or after a target marker variant according to a particular order. In particular, an adjacent marker variant includes a marker variant represented by an adjacent column that is positioned one column before or one column after a target column representing a target marker variant within a matrix. As explained further below, in some cases, an adjacent marker variant is represented as m−1 or [m−1] or as m+1 or [m+1].

Relatedly, as used herein, the term “adjacent-marker intermediate allele likelihood” refers to an intermediate allele likelihood for a marker variant that is adjacent to a target marker variant. In particular, an adjacent-marker intermediate allele likelihood includes an intermediate allele likelihood for a marker variant represented by an adjacent column that is positioned one column before or one column after a target column representing a target marker variant within a matrix. As explained further below, in some cases, an adjacent-marker intermediate allele likelihood is represented as A[m−1][k].

As further used herein, the term “allele-likelihood factor” refers to a factor or parameter that corresponds to a haplotype allele and that is applied to a transition coefficient and/or other parameters in a function. In particular, an allele-likelihood factor includes a factor or parameter that (i) corresponds to either a sample reference haplotype allele or a sample alternate haplotype allele and a marker variant and (ii) is applied to a transition linear coefficient, a transition constant coefficient, and/or other parameters in a function to determine an allele likelihood. As explained further below, in some cases, an allele-likelihood factor is generally represented as Q[m][Allele], an allele-likelihood factor corresponding to a sample reference haplotype allele is represented as Q0, and an allele-likelihood factor corresponding to a sample alternate haplotype allele is represented as Q1.

Relatedly, the term “transition coefficient” refers to a coefficient or parameter representing a probability of transitioning or changing between marker variants. In particular, a transition coefficient includes a coefficient or parameter representing a probability of transitioning between rows representing marker variants within a matrix. In some cases, a transition coefficient comes in a couple of varieties, including a transition linear coefficient and a transition constant coefficient. As described below, in some cases, a transition constant coefficient is represented as P0, and a transition linear coefficient is represented as P1.

In some cases, the accelerated genotype-imputation system combines (e.g., multiplies, weighted sums) various factors or coefficients. For example, as used herein, the term “transition-aware allele-likelihood factor” refers to a value representing a combination of a transition coefficient and an allele likelihood factor. In particular, a transition-aware allele-likelihood factor includes a value representing a product of a transition coefficient and an allele likelihood factor. As described below, in some cases, a transition-aware allele-likelihood factor is generally represented as Q[m][Allele]*P[m], a first transition-aware allele-likelihood factor is represented as Q[m][Allele]*P1[m], and a second transition-aware likelihood factor is represented as Q[m][Allele]*P0[m].

As further used herein, the term “adjacent-marker-transition-factor-aware allele likelihood” refers to a value representing a combination of an allele likelihood factor, a transition coefficient, and an intermediate allele likelihood for an adjacent marker variant. In particular, an adjacent-marker-transition-factor-aware allele likelihood includes a value representing a product of an allele likelihood factor, a transition linear coefficient, and an intermediate allele likelihood for an adjacent marker variant. As described below, in some cases, an adjacent-marker-transition-factor-aware allele likelihood is generally represented as Q[m][Allele]*P1[m]*A′[m−1].

As further used herein, the term “summed-adjacent-marker transition-aware allele-likelihood factor” refers to a value representing a combination of an allele likelihood factor, a transition coefficient, and a sum of intermediate allele likelihoods for an adjacent marker variant. In particular, a summed-adjacent-marker transition-aware allele-likelihood factor includes a value representing a product of an allele likelihood factor, a transition constant coefficient, and a sum of intermediate allele likelihoods for an adjacent marker variant. As described below, in some cases, a summed-adjacent-marker transition-aware allele-likelihood factor is generally represented as Q[m][Allele]*P0[m]*Sum′[m−1].

As indicated above, in some embodiments, the accelerated genotype-imputation system can extemporaneously generate sets of intermediate allele likelihoods for multiple passes by using the intermediate-allele-likelihood subsets as hot-start points for a full pass of intermediate allele likelihoods. As used herein, the term “pass” refers to a sequence of operations to determine intermediate allele likelihoods corresponding to haplotypes from a haplotype reference panel according to a particular direction. In particular, a pass includes a sequence of operations in a direction across a haplotype matrix to determine intermediate allele likelihoods corresponding to different combinations of marker variants and haplotypes from a haplotype reference panel. For example, a pass may proceed in a forward or reverse direction across a haplotype matrix. In some cases, a pass that includes a sequence of operations from left to right of a haplotype matrix constitutes an alpha pass, and a pass that includes a sequence of operations from right to left of the haplotype matrix constitutes a beta pass.

Relatedly, the phrase “pass intermediate allele likelihoods” refers to a set of intermediate allele likelihoods corresponding to a pass. In particular, a set of first-pass intermediate allele likelihoods includes a set of intermediate allele likelihoods determined by performing a first pass of operations in a first direction. By contrast, a set of second-pass intermediate allele likelihoods includes a set of intermediate allele likelihoods determined by performing a second pass of operations in a second direction. For instance, a set of first-pass intermediate allele likelihoods may be determined when the accelerated genotype-imputation system performs a first pass in a backward direction across a haplotype matrix, and a set of second-pass intermediate allele likelihoods may be determined when the accelerated genotype-imputation system performs a second pass in a forward direction across the haplotype matrix, or vice versa.

As indicated above, in some embodiments, the accelerated genotype-imputation system stores or accesses a subset of first-pass or second-pass intermediate allele likelihoods corresponding to a subset of marker variants for groups of marker variants. As used herein, the term “group of marker variants” refers to a segment or window of marker variants from among a larger set of marker variants. For instance, groups of marker variants may include multiple groups of 100, 1,000, or 5,000 consecutively ordered marker variants among a set of 50,000 marker variants. Because a haplotype matrix may represent a set of marker variants by columns, where each individual column represents an individual marker variant, a group of marker variants may likewise correspond to a group of rows. Accordingly, a subset of first-pass or second-pass intermediate allele likelihoods corresponding to a subset of marker variants may refer to a subset that includes one intermediate allele likelihood for one marker variant from among each group of marker variants, such as 1 marker variant for every 100, 1,000, or 5,000 marker variants.

As further indicated above, in some embodiments, the accelerated genotype-imputation system determines different running sums of different subsets of intermediate allele likelihoods of the genomic region comprising different types of haplotype alleles. As used herein, the term “running sum of a subset of intermediate allele likelihoods” refers to a summed value of one or more intermediate allele likelihoods for a marker variant (e.g., an adjacent marker variant) that can be updated as additional intermediate allele likelihoods are determined. In particular, a running sum of a subset of intermediate allele likelihoods includes a summed value of multiple intermediate allele likelihoods of a genomic region exhibiting or comprising a particular type of haplotype allele from one or more haplotype of a haplotype reference panel—given an adjacent marker variant—where the summed value can be updated as additional intermediate allele likelihoods corresponding to the adjacent marker variant are determined. Accordingly, in some embodiments, the accelerated genotype-imputation system (i) determines, for an adjacent marker variant, a running sum of a first subset of intermediate allele likelihoods of the genomic region comprising a first type of haplotype allele (e.g., a sample reference haplotype allele) from one or more haplotypes of the haplotype reference panel and (ii) determines, for the adjacent marker variant, a running sum of a second subset of intermediate adjacent-allele likelihoods of the genomic region comprising a second type of haplotype allele (e.g., a sample alternate haplotype allele) from the one or more haplotypes.

Additionally, as used herein, the term “genomic coordinate” refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleotide-base within the source for the reference genome (e.g., mt:16568 or SARS-CoV-2:29001). By contrast, in certain cases, a genomic coordinate refers to a position of a nucleotide-base within a reference genome without reference to a chromosome or source (e.g., 29727).

Further, as used herein, a “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870).

As used herein, for example, the term “configurable processor” refers to a circuit or chip that can be configured or customized to perform a specific application. For instance, a configurable processor includes an integrated circuit chip that is designed to be configured or customized on site by an end user's computing device to perform a specific application. Configurable processors include, but are not limited to, an ASIC, ASSP, a coarse-grained reconfigurable array (CGRA), or FPGA. By contrast, configurable processors do not include a CPU or GPU. In some embodiments, the accelerated genotype-imputation system uses a configurable processor (e.g., FPGA) or a processor (e.g., CPU) to perform the various embodiments described herein.

As further used herein, the term “nucleobase call” (or simply “base call”) refers to a determination or prediction of a particular nucleobase (or nucleobase pair) for an oligonucleotide (e.g., read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide-fragment read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or other base-call-output file-based on nucleotide-fragment reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, or a thymine (T) call.

As further used herein, the term “nucleotide-sample slide” refers to a plate or slide comprising oligonucleotides for sequencing nucleotide sequences from genomic samples or other sample nucleic-acid polymers. In particular, a nucleotide-sample slide can refer to a slide containing fluidic channels through which reagents and buffers can travel as part of sequencing. For example, in one or more embodiments, a nucleotide-sample slide includes a flow cell (e.g., a patterned flow cell or non-patterned flow cell) comprising small fluidic channels and short oligonucleotides complementary to binding adapter sequences. As indicated above, a nucleotide-sample slide can include wells (e.g., nanowells) comprising clusters of oligonucleotides.

As suggested above, a flow cell or other nucleotide-sample slide can (i) include a device having a lid extending over a reaction structure to form a flow channel therebetween that is in communication with a plurality of reaction sites of the reaction structure and (ii) include a detection device that is configured to detect designated reactions that occur at or proximate to the reaction sites. A flow cell or other nucleotide-sample slide may include a solid-state light detection or imaging device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDS)). The excitation light may provide emission signals (e.g., light of a wavelength or wavelengths that differ from the excitation light and, potentially, each other) that may be detected by the light sensors of the flow cell.

As further used herein, the term “sequencing run” refers to an iterative process on a sequencing device to determine a primary structure of nucleotide sequences from a sample (e.g., genomic sample). In particular, a sequencing run includes cycles of sequencing chemistry and imaging performed by a sequencing device that incorporate nucleobases into growing oligonucleotides to determine nucleotide-fragment reads from nucleotide sequences extracted from a sample (or other sequences within a library fragment) and seeded throughout a nucleotide-sample slide. In some cases, a sequencing run includes replicating nucleotide sequences from one or more genome samples seeded in clusters throughout a nucleotide-sample slide (e.g., a flow cell). Upon completing a sequencing run, a sequencing device can generate base-call data in a file.

As just suggested, the term “base-call data” refers to data representing nucleobase calls for nucleotide-fragment reads and/or corresponding sequencing metrics. For instance, base-call data includes textual data representing nucleobase calls for nucleotide-fragment reads as text (e.g., A, C, G, T) along with corresponding base-call-quality metrics, depth metrics, and/or other sequencing metrics. In some cases, base-call data is formatted in a text file, such as a binary base call (BCL) sequence file or as a fast-all quality (FASTQ) file.

As further used herein, the term “nucleotide-fragment read” (or simply “read”) refers to an inferred sequence of one or more nucleobases (or nucleobase pairs) from all or part of a sample nucleotide sequence (e.g., a sample genomic sequence, cDNA). In particular, a nucleotide-fragment read includes a determined or predicted sequence of nucleobase calls for a nucleotide sequence (or group of monoclonal nucleotide sequences) from a sample library fragment corresponding to a genome sample. For example, in some cases, a sequencing device determines a nucleotide-fragment read by generating nucleobase calls for nucleobases passed through a nanopore of a nucleotide-sample slide, determined via fluorescent tagging, or determined from a cluster in a flow cell.

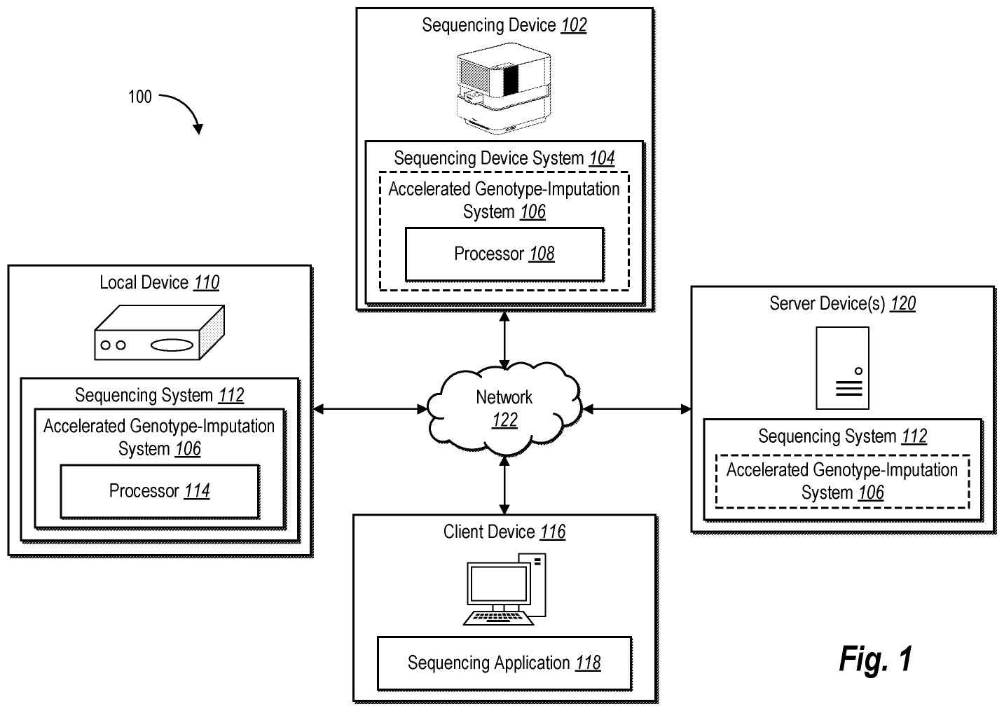

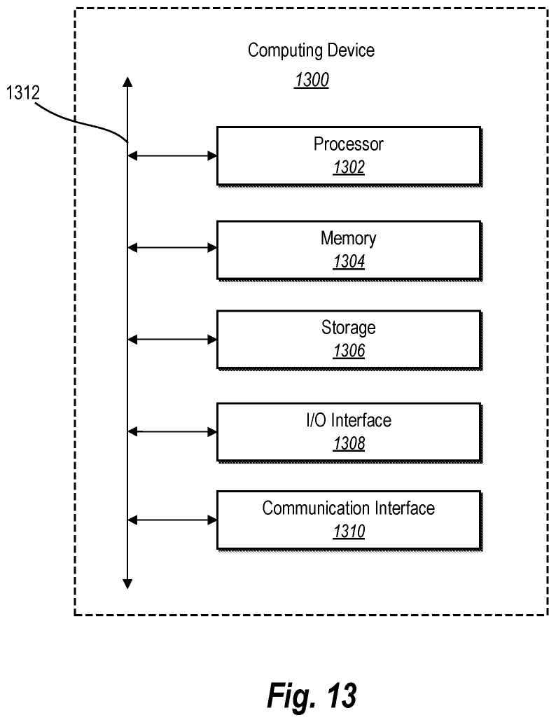

The following paragraphs describe the accelerated genotype-imputation system with respect to illustrative figures that portray example embodiments and implementations. For example, FIG. 1 illustrates a schematic diagram of a computing system 100 in which an accelerated genotype-imputation system 106 operates in accordance with one or more embodiments. As illustrated, the computing system 100 includes a sequencing device 102 connected to a local device 110 (e.g., a local server device), one or more server device(s) 120, and a client device 116. As shown in FIG. 1, the sequencing device 102, the local device 110, the server device(s) 120, and the client device 116 can communicate with each other via a network 122. The network 122 comprises any suitable network over which computing devices can communicate. Example networks are discussed in additional detail below with respect to FIG. 13. While FIG. 1 shows an embodiment of the accelerated genotype-imputation system 106, this disclosure describes alternative embodiments and configurations below.

As indicated by FIG. 1, the sequencing device 102 comprises a computing device and a sequencing device system 104 for sequencing a genomic sample or other nucleic-acid polymer. In some embodiments, by executing the sequencing device system 104 on a processor 108 (e.g., a configurable processor), the sequencing device 102 analyzes nucleotide fragments or oligonucleotides extracted from genomic samples to generate nucleotide-fragment reads or other data utilizing computer implemented methods and systems either directly or indirectly on the sequencing device 102. More particularly, the sequencing device 102 receives nucleotide-sample slides (e.g., flow cells) comprising nucleotide fragments extracted from samples and further copies and determines the nucleobase sequence of such extracted nucleotide fragments.

In one or more embodiments, the sequencing device 102 utilizes SBS to sequence nucleotide fragments into nucleotide-fragment reads and determine nucleobase calls for the nucleotide-fragment reads. In addition or in the alternative to communicating across the network 122, in some embodiments, the sequencing device 102 bypasses the network 122 and communicates directly with the local device 110 or the client device 116. By executing the sequencing device system 104, the sequencing device 102 can further store the nucleobase calls as part of base-call data that is formatted as a BCL file and send the BCL file to the local device 110 and/or the server device(s) 120.

As further indicated by FIG. 1, the local device 110 is located at or near a same physical location of the sequencing device 102. Indeed, in some embodiments, the local device 110 and the sequencing device 102 are integrated into a same computing device. The local device 110 may run a sequencing system 112 to generate, receive, analyze, store, and transmit digital data, such as by receiving base-call data or determining variant calls based on analyzing such base-call data. As shown in FIG. 1, the sequencing device 102 may send (and the local device 110 may receive) base-call data generated during a sequencing run of the sequencing device 102. By executing software in the form of the sequencing system 112, the local device 110 may align nucleotide-fragment reads with a reference genome and determine genetic variants based on the aligned nucleotide-fragment reads. The local device 110 may also communicate with the client device 116. In particular, the local device 110 can send data to the client device 116, including a variant call file (VCF) or other information indicating nucleobase calls, sequencing metrics, error data, or other metrics.

As indicated above, as part of the local device 110, the accelerated genotype-imputation system 106 can determine intermediate allele likelihoods of a genomic region exhibiting certain haplotype alleles as part of a genotype imputation model by using one or both of consolidated computations and data exchanges across specialized hardware. For instance, the accelerated genotype-imputation system 106 can determine an intermediate allele likelihood of a genomic region comprising a haplotype allele—given a particular marker variant and a haplotype from a haplotype reference panel—by running a single, pass-concurrent multiplication operation on a processor 114. In certain implementations, the processor 114 is a configurable processor. In some cases, the accelerated genotype-imputation system 106 (i) determines and stores subsets of intermediate allele likelihoods corresponding to groups of marker variants and (ii) extemporaneously generate sets of intermediate allele likelihoods for multiple passes by using the intermediate-allele-likelihood subsets as hot-start points for a full pass of intermediate allele likelihoods. In further embodiments, the accelerated genotype-imputation system 106 determines running sums of intermediate allele likelihoods of a genomic region exhibiting haplotype alleles for one or more haplotypes given on marker variant as running inputs for determining intermediate allele likelihoods of the genomic region exhibiting the haplotype alleles given another marker variant.

As further indicated by FIG. 1, the server device(s) 120 are located remotely from the local device 110 and the sequencing device 102. Similar to the local device 110, in some embodiments, the server device(s) 120 include a version of the sequencing system 112. Accordingly, the server device(s) 120 may generate, receive, analyze, store, and transmit digital data, such as by receiving base-call data or determining variant calls based on analyzing such base-call data. Accordingly, the sequencing device 102 may send (and the server device(s) 120 may receive) base-call data from the sequencing device 102. The server device(s) 120 may also communicate with the client device 116. In particular, the server device(s) 120 can send data to the client device 116, including VCFs or other sequencing related information.

In some embodiments, the server device(s) 120 comprise a distributed collection of servers where the server device(s) 120 include a number of server devices distributed across the network 122 and located in the same or different physical locations. Further, the server device(s) 120 can comprise a content server, an application server, a communication server, a web-hosting server, or another type of server.

As further illustrated and indicated in FIG. 1, by executing a sequencing application 118, the client device 116 can generate, store, receive, and send digital data. In particular, the client device 116 can receive sequencing data from the local device 110 or receive call files (e.g., BCL) and sequencing metrics from the sequencing device 102. Furthermore, the client device 116 may communicate with the local device 110 or the server device(s) 120 to receive a VCF comprising nucleobase calls and/or other metrics, such as a base-call-quality metrics or pass-filter metrics. The client device 116 can accordingly present or display information pertaining to variant calls or other nucleobase calls within a graphical user interface of the sequencing application 118 to a user associated with the client device 116. For example, the client device 116 can present variant calls and/or sequencing metrics for a sequenced genomic sample within a graphical user interface of the sequencing application 118.

Although FIG. 1 depicts the client device 116 as a desktop or laptop computer, the client device 116 may comprise various types of client devices. For example, in some embodiments, the client device 116 includes non-mobile devices, such as desktop computers or servers, or other types of client devices. In yet other embodiments, the client device 116 includes mobile devices, such as laptops, tablets, mobile telephones, or smartphones. Additional details regarding the client device 116 are discussed below with respect to FIG. 13.

As further illustrated in FIG. 1, the client device 116 includes the sequencing application 118. The sequencing application 118 may be a web application or a native application stored and executed on the client device 116 (e.g., a mobile application, desktop application). The sequencing application 118 can include instructions that (when executed) cause the client device 116 to receive data from the accelerated genotype-imputation system 106 and present, for display at the client device 116, base-call data or data from a VCF. Furthermore, the sequencing application 118 can instruct the client device 116 to display summaries for multiple sequencing runs.

As further illustrated in FIG. 1, a version of the accelerated genotype-imputation system 106 may be located and implemented (e.g., entirely or in part) on the local device 110. In yet other embodiments, the accelerated genotype-imputation system 106 is implemented by one or more other components of the computing system 100, such as the server device(s) 120. In particular, the accelerated genotype-imputation system 106 can be implemented in a variety of different ways across the sequencing device 102, the local device 110, the server device(s) 120, and the client device 116. For example, the accelerated genotype-imputation system 106 can be downloaded from the server device(s) 120 to the accelerated genotype-imputation system 106 and/or the local device 110 where all or part of the functionality of the accelerated genotype-imputation system 106 is performed at each respective device within the computing system 100.

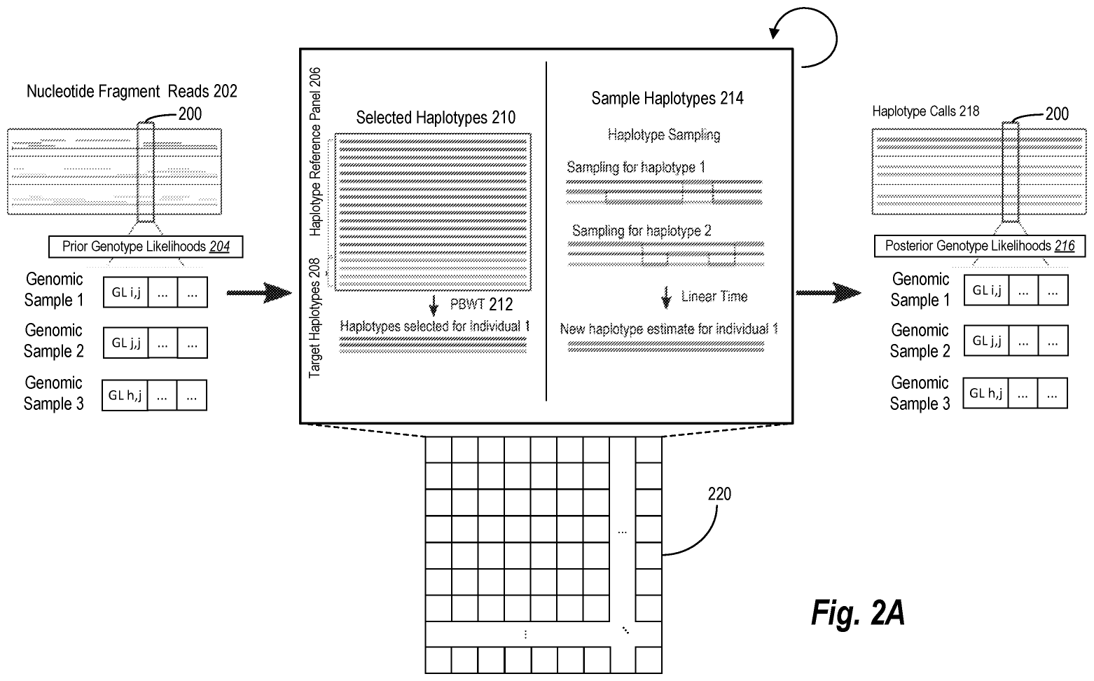

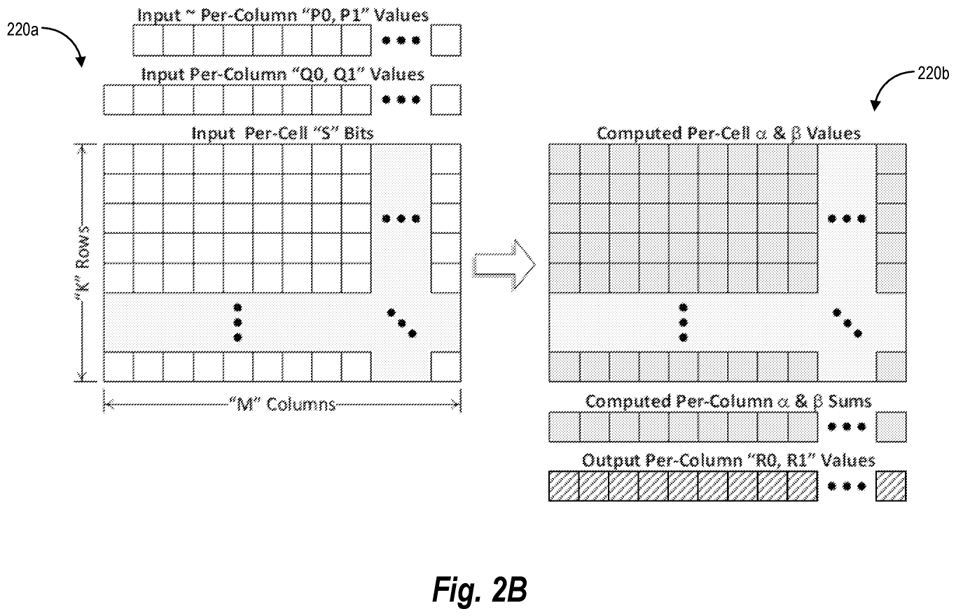

As suggested above, in some embodiments, the accelerated genotype-imputation system 106 applies a genotype imputation model, such as a hidden Markov model (HMM)-based genotype imputation model, to nucleotide-fragment reads corresponding to a genomic region of a genomic sample. By applying a genotype imputation model, the accelerated genotype-imputation system 106 can determine posterior genotype likelihoods and haplotype calls for the genomic region. In accordance with one or more embodiments, FIG. 2A illustrates the accelerated genotype-imputation system 106 applying GLIMPSE as a genotype imputation model to determine posterior genotype likelihoods for a genomic region of multiple genomic samples. As part of imputing haplotypes using an HMM, the accelerated genotype-imputation system 106 utilizes a haplotype matrix 220 to determine haplotype allele likelihoods corresponding to the genomic region. In accordance with one or more embodiments, FIG. 2B illustrates a more detailed depiction of the accelerated genotype-imputation system 106 utilizing the haplotype matrix 220 to determine such haplotype allele likelihoods.

As shown in FIG. 2A, for instance, the accelerated genotype-imputation system 106 determines prior genotype likelihoods 204 that genomic regions 200 from multiple genomic samples exhibit particular genotypes (e.g., a reference allele or alternate allele). As suggested by FIG. 2A, in some cases, the genomic regions 200 corresponding to an approximately same set of genomic coordinates (with respect to a reference genome) for the multiple genomic samples. As indicated by nucleotide-fragment reads 202, the genomic regions 200 exhibit low coverage (e.g., ≤8× read coverage). In some embodiments, the accelerated genotype-imputation system 106 uses a probabilistic call generation model (e.g., variant caller from DRAGEN) to determine the prior genotype likelihoods 204 based on (i) the nucleotide-fragment reads 202 from the multiple genomic samples and (i) quality scores for base calls of the nucleotide-fragment reads 202.

As further indicated by FIG. 2A, the genomic regions 200 correspond to variable positions (or variable genomic coordinates) of a haplotype reference panel 206. The accelerated genotype-imputation system 106 further deconvolves a vector of the prior genotype likelihoods 204 to two independent vectors of haplotype allele likelihoods (or, simply, haplotype likelihoods), where each vector corresponds to one of two complementary haplotypes. In some such embodiments, the accelerated genotype-imputation system 106 inputs the prior genotype likelihoods 204 in vector form as part of an input matrix.

Based on the haplotype likelihoods from the independent vectors, in some implementations, the accelerated genotype-imputation system 106 imputes two target haplotypes as haplotype calls using a haploid version of an HMM in an iterative process. As shown in FIG. 2A, for instance, the accelerated genotype-imputation system 106 selects haplotypes 210 based on the haplotype reference panel 206 and target haplotypes 208 estimated for each genomic sample. After selecting haplotypes for a given genomic sample, the accelerated genotype-imputation system 106 stores reference and target versions of the selected haplotypes as a Positional Burrows Wheeler Transform (PBWT) 212.

As further shown in FIG. 2A, in some embodiments, the accelerated genotype-imputation system 106 samples haplotypes 214 in the PBWT 212 format by performing a linear-time-sampling algorithm based on a haplotype imputation version of HMM developed by Na Li and Matthew Stephens, “Modeling Linkage Disequilibrium and Identifying Recombination Hotspots Using Single-Nucleotide Polymorphism Data,” 165 Genetics 2213-2233 (2003), which is hereby incorporated by reference in its entirety. By performing the linear-time-sampling algorithm as part of sampler iterations, the accelerated genotype-imputation system 106 further determines (and updates) the phase of two imputed haplotypes for a genomic region of the genomic regions 200 for a particular genomic sample.

Based on the imputed and phased haplotypes, as further shown in FIG. 2A, the accelerated genotype-imputation system 106 determines posterior genotype likelihoods 216 that the genomic regions 200 of multiple genomic samples exhibit particular genotypes (e.g., a reference allele or alternate allele). The accelerated genotype-imputation system 106 further determines haplotype calls 218 for the genomic region for each of the multiple genomic samples. As indicated above, in some embodiments, the accelerated genotype-imputation system 106 uses a modified version of GLIMPSE developed by Rubinacci as a genotype imputation model.

As part of selecting haplotypes 210 and sampling haplotypes 214, the accelerated genotype-imputation system 106 can perform sampler iterations across genomic samples using a haplotype matrix 220. As explained further below and as depicted further in FIG. 2B, the accelerated genotype-imputation system 106 can determine intermediate allele likelihoods of genomic regions comprising haplotype alleles in both a forward and reverse direction across the haplotype matrix 220. In the haplotype matrix 220, each column represents a marker variant and each row represents a haplotype from the haplotype reference panel 206. The accelerated genotype-imputation system 106 further determines a sum of the intermediate allele likelihoods for each column representing a marker variant. Based on the summed adjacent-marker intermediate allele likelihoods for each column, in some cases, the accelerated genotype-imputation system 106 determines allele likelihoods for the corresponding marker variant and haplotypes. Such allele likelihoods represent an example or an embodiment of the posterior genotype likelihoods 216.

As shown in FIG. 2B, for instance, the accelerated genotype-imputation system 106 uses an input haplotype matrix 220a to input various values. As depicted in FIG. 2B, the input haplotype matrix 220a and an updated haplotype matrix 220b are organized by “K” rows representing haplotypes from the haplotype reference panel 206 and by “M” columns representing marker variants (e.g., SNPs or other variants). Accordingly, each row represents a haplotype “k,” and each column represents a marker variant “m.” In some embodiments, both the input haplotype matrix 220a and the updated haplotype matrix 220b include approximately 1,000 rows representing approximately 1,000 haplotypes from the haplotype reference panel 206 and approximately 50,000 columns representing approximately 50,000 marker variants. Accordingly, the input haplotype matrix 220a includes approximately 50 million cells. But other suitable dimensions may be used of greater or fewer columns and rows.

As further indicated by FIG. 2B, in some embodiments, the accelerated genotype-imputation system 106 inputs values for transition coefficients (e.g., P0 and P1) and allele-likelihood factors (e.g., Q0 and Q1) into each cell of the input haplotype matrix 220a. For instance, the accelerated genotype-imputation system 106 inputs into each cell a particular transition linear coefficient (e.g., P1) and a particular transition constant coefficient (e.g., P0), where transition coefficients generally represent probabilities of transitioning between haplotypes represented by neighboring rows. Further, the accelerated genotype-imputation system 106 inputs into each cell a particular allele-likelihood factor (e.g., Q0) for a first type of haplotype allele for a particular haplotype represented by a row and inputs a particular allele-likelihood factor (e.g., Q1) for a second type of haplotype allele of the particular haplotype represented by the row. As noted above, in some embodiments, one allele-likelihood factor (e.g., Q0) corresponds to a sample reference haplotype allele of a particular haplotype represented by a row, and another allele-likelihood factor (e.g., Q1) corresponds to a sample alternate haplotype of the particular haplotype.

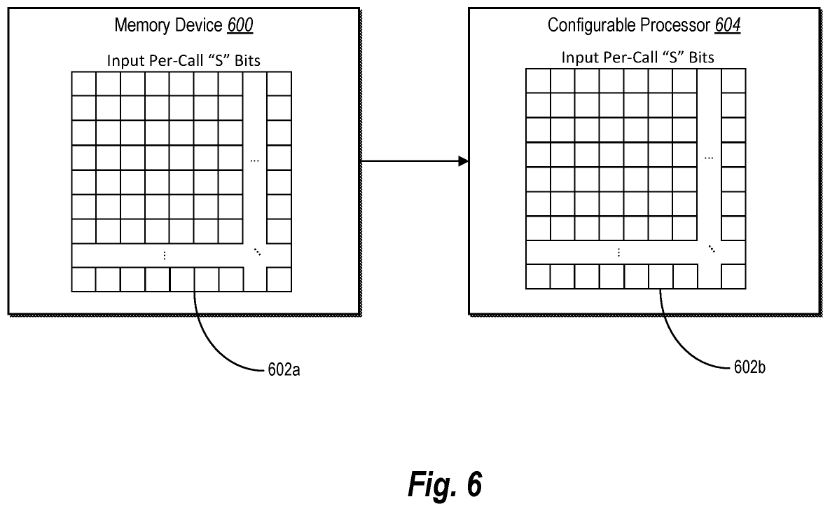

In addition to inputting transition coefficients and allele-likelihood factors, as further shown in FIG. 2B, in certain embodiments, the accelerated genotype-imputation system 106 inputs values representing haplotype alleles (S bits) into each cell of the input haplotype matrix 220a. In particular, the accelerated genotype-imputation system 106 can input a value (or a bit) of 0 indicating a sample reference haplotype allele of a particular haplotype represented by a row. Conversely, the accelerated genotype-imputation system 106 can input a value (or a bit) of 1 indicating a sample alternate haplotype allele of the particular haplotype represented by the row. For brevity, this disclosure refers to such input values representing haplotype alleles as haplotype-allele-indicator data for a haplotype matrix, as further described below with respect to FIG. 6.

After inputting values for the transition coefficients, allele-likelihood factors, and haplotype-allele indicators, in some embodiments, the accelerated genotype-imputation system 106 determines an intermediate allele likelihood in each cell based on the input values. For example, in some embodiments, the accelerated genotype-imputation system 106 performs an alpha pass and a beta pass across the cells of the input haplotype matrix 220a to determine intermediate allele likelihoods represented by darker shading in the updated haplotype matrix 220b. Indeed, in certain embodiments, the alpha values represent intermediate allele likelihoods (e.g., A[m][k]) determined during an alpha pass, and the beta values represent intermediate allele likelihoods (e.g., A[m][k]) determined during a beta pass. As further described below, in some embodiments, the accelerated genotype-imputation system 106 performs two beta passes (including a sacrificial bet pass) as part of an HMM-computation task.

To determine an intermediate allele likelihood (e.g., A[m][k]) for a target cell, in some embodiments, the accelerated genotype-imputation system 106 determines a first product of a transition linear coefficient for a target marker variant (e.g., P1 [m]), a normalization value for a column representing an adjacent marker variant (e.g., Norm[m−1]), and an adjacent-marker intermediate allele likelihood for an adjacent marker variant (e.g., A[m−1][k]). The normalization value for a given marker variant (e.g., represented by a column) can by any value that facilitates keeping per-cell values from overflowing the number representation in which an intermediate-allele-likelihood value or sum of intermediate-allele-likelihood values exist. The accelerated genotype-imputation system 106 further determines a second product of a transition constant coefficient (e.g., P0[m]), a normalization value for the column representing an adjacent marker variant (e.g., Norm[m−1]), and summed adjacent-marker intermediate allele likelihoods for the adjacent marker variant (e.g., Sum[m−1]). The accelerated genotype-imputation system 106 further multiplies a sum of the first product and the second product by an allele-likelihood factor (e.g., Q[m][Allele]) to determine the intermediate allele likelihood for the target cell.

As noted above, such an allele-likelihood factor may constitute an allele-likelihood factor (e.g., Q0) corresponding to a sample reference haplotype allele of a particular haplotype represented by a row or another allele-likelihood factor (e.g., Q1) corresponds to a sample alternate haplotype of the particular haplotype. As described below, however, the accelerated genotype-imputation system 106 can also perform an improved way of determining such an intermediate allele likelihood.

As further shown in FIG. 2B, in some embodiments, the accelerated genotype-imputation system 106 determines, for each column, a sum of alpha values for a marker variant and a sum of beta values for the marker variant. In particular, in some embodiments, the accelerated genotype-imputation system 106 determines (i) a sum of intermediate allele likelihoods for a column represented by a marker variant in one pass and (ii) a sum of intermediate allele likelihoods for the column represented by the marker variant in another pass.

Based on the summed intermediate allele likelihoods for each marker variant represented by a column, in some embodiments shown in FIG. 2B, the accelerated genotype-imputation system 106 further determines a pair of allele likelihoods (e.g., R0 and R1) for each marker variant. For instance, in certain implementations, the accelerated genotype-imputation system 106 determines first allele likelihoods (e.g., R0) that a genomic region comprises a sample reference haplotype allele corresponding to various haplotypes represented by the various rows. Similarly, the accelerated genotype-imputation system 106 determines second allele likelihoods (e.g., R1) that a genomic region comprises a sample alternate haplotype allele corresponding to various haplotypes represented by the various rows.

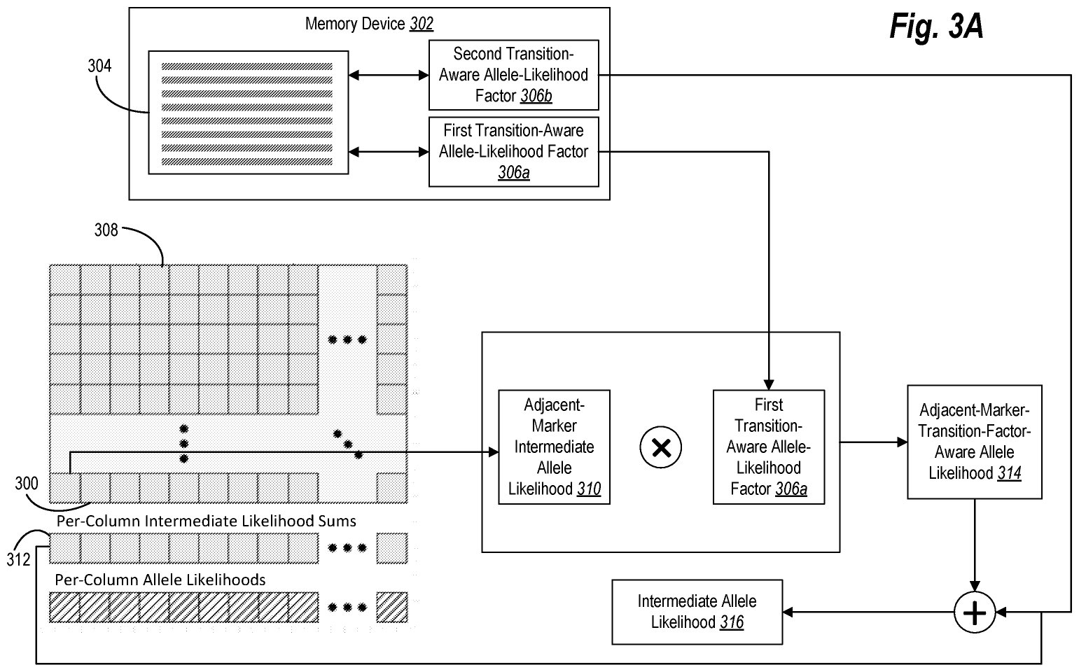

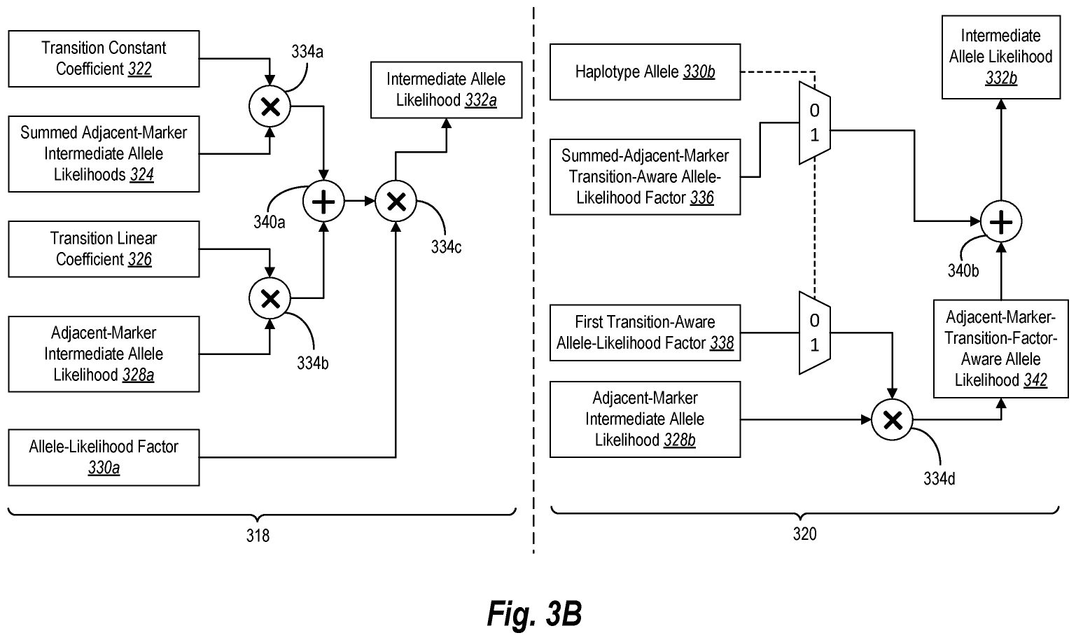

As noted above, in some cases, the accelerated genotype-imputation system 106 expedites intermediate-allele-likelihood determinations by performing a single, pass-concurrent multiplication operation for a given a target marker variant and haplotype from a haplotype reference panel. In accordance with one or more embodiments, FIG. 3A depicts the accelerated genotype-imputation system 106 running a single, pass-concurrent multiplication operation to determine an intermediate allele likelihood of a genomic region comprising a haplotype allele—given a target cell representing a target marker variant and a target haplotype from a haplotype reference panel. FIG. 3B depicts a comparison of the accelerated genotype-imputation system 106 determining such an intermediate allele likelihood for a target cell using either (i) three pass-concurrent multiplication operations or (ii) one pass-concurrent multiplication operation. By predetermining transition-aware allele-likelihood factors before a processor determines intermediate allele likelihoods for a target marker variant, the accelerated genotype-imputation system 106 condenses and expedites the processing load from three pass-concurrent multiplication operations to one pass-concurrent multiplication operation for a target cell.

As shown in FIG. 3A, for instance, the accelerated genotype-imputation system 106 identifies, from within a memory device 302, a haplotype reference panel 304 corresponding to a genomic region of one or more genomic samples and transition-aware allele-likelihood factors to perform a genotype imputation model. In particular, in some embodiments, the accelerated genotype-imputation system 106 identifies the haplotype reference panel 304 stored on dynamic random-access memory (DRAM), dynamic random-access memory (SRAM), or a cache memory device. Further, the accelerated genotype-imputation system 106 identifies a first transition-aware allele-likelihood factor 306a and a second transition-aware allele-likelihood factor 306b while performing an alpha or beta pass of a haplotype matrix 308. In some cases, the accelerated genotype-imputation system 106 identifies the first transition-aware allele-likelihood factor 306a and the second transition-aware allele-likelihood factor 306b upon arriving at a target cell 300 representing a combination of a target marker variant and a haplotype during a pass of the haplotype matrix 308.

To avoid determining the first and second transition-aware allele-likelihood factors 306a and 306b during a pass, in some embodiments, the accelerated genotype-imputation system 106 predetermines the first and second transition-aware allele-likelihood factors 306a and 306b before determining intermediate allele likelihoods for a column representing a target marker variant within the haplotype matrix 308. To predetermine the first transition-aware allele-likelihood factor 306a, in some embodiments, the accelerated genotype-imputation system 106 combines (e.g., multiplies, weighted sums) an allele-likelihood factor for a haplotype allele and a transition constant coefficient for transitioning between haplotypes from the haplotype reference panel 304. Similarly, to predetermine the second transition-aware allele-likelihood factor 306b, the accelerated genotype-imputation system 106 combines (e.g., multiplies, weighted sums) the allele-likelihood factor and a transition linear coefficient for transitioning between haplotypes from the haplotype reference panel 304.

The accelerated genotype-imputation system 106 can generate predetermined versions of the first and second transition-aware allele-likelihood factors 306a and 306b because input values are available before a pass across the haplotype matrix 308 or at least before determining intermediate allele likelihoods for a target marker variant. Because the accelerated genotype-imputation system 106 has access to (and can identify) allele-likelihood factors and transition coefficients for a column representing the target marker variant before determining intermediate allele likelihoods for a target marker variant, in certain implementations, the accelerated genotype-imputation system 106 generates predetermined versions of the first and second transition-aware allele-likelihood factors 306a and 306b. Accordingly, in some embodiments, the accelerated genotype-imputation system 106 predetermines the first and second transition-aware allele-likelihood factors 306a and 306b before determining one or more intermediate allele likelihoods corresponding to the marker variant as part of a pass of the haplotype matrix 308.

As part of performing a pass of determining intermediate allele likelihoods, in certain cases, the accelerated genotype-imputation system 106 determines and accesses values as part of the pass across the haplotype matrix 308. To determine an intermediate allele likelihood 316 for the target cell 300, in certain embodiments, the accelerated genotype-imputation system 106 identifies, from the haplotype matrix 308, the adjacent-marker intermediate allele likelihood 310 for an adjacent marker variant to the target marker variant. In the haplotype matrix 308, an adjacent column represents the adjacent marker variant next to a target column that represents the target marker variant. As part of a pass across the haplotype matrix 308, in some embodiments, the accelerated genotype-imputation system 106 determines the adjacent-marker intermediate allele likelihood 310 for a combination of the adjacent marker variant and the target haplotype from the haplotype reference panel 304 before determining the intermediate allele likelihood 316.

After identifying the relevant input values for a multiplication operation, as further shown in FIG. 3A, the accelerated genotype-imputation system 106 combines the adjacent-marker intermediate allele likelihood 310 and the first transition-aware allele-likelihood factor 306a. In particular, in some embodiments, the accelerated genotype-imputation system 106 multiplies the adjacent-marker intermediate allele likelihood 310 and the first transition-aware allele-likelihood factor 306a during a pass of the haplotype matrix 308. Because the accelerated genotype-imputation system 106 determines both the adjacent-marker intermediate allele likelihood 310 and the first transition-aware allele-likelihood factor 306a before passing a cell representing the target marker variant and the target haplotype, the accelerated genotype-imputation system 106 can use this single, pass-concurrent multiplication operation as part of determining the intermediate allele likelihood 316 for the target cell 300. Based on combining the adjacent-marker intermediate allele likelihood 310 and the first transition-aware allele-likelihood factor 306a, as shown in FIG. 3A, the accelerated genotype-imputation system 106 generates an adjacent-marker-transition-factor-aware allele likelihood 314.

As further suggested above, in some embodiments, the accelerated genotype-imputation system 106 determines the intermediate allele likelihood 316 of the genomic region comprising a haplotype allele based on the adjacent-marker-transition-factor-aware allele likelihood 314 and the second transition-aware allele-likelihood factor 306b. For instance, in some embodiments, the accelerated genotype-imputation system 106 determines a sum of the adjacent-marker-transition-factor-aware allele likelihood 314 and the second transition-aware allele-likelihood factor 306b to determine the intermediate allele likelihood 316. As explained further below, in certain implementations, the accelerated genotype-imputation system 106 determines the intermediate allele likelihood 316 by determining a sum of (i) the adjacent-marker-transition-factor-aware allele likelihood 314 and (ii) a product of the second transition-aware allele-likelihood factor 306b and summed adjacent-marker intermediate allele likelihoods 312 for the adjacent marker variant.

As indicated above, the accelerated genotype-imputation system 106 can reduce computer processing from three multiplication operations to one multiplication operation to determine an intermediate allele likelihood for a target cell. In accordance with one or more embodiments, FIG. 3B depicts the accelerated genotype-imputation system 106 using a configurable processor to perform a multiple-multiplication model 318 and a single-multiplication model 320 for determining an intermediate allele likelihood for a target cell representing a combination of a target marker variant and a haplotype within a haplotype matrix.

As shown in FIG. 3B, the accelerated genotype-imputation system 106 performs multiplication operations 334a, 334b, and 334c as part of determining an intermediate allele likelihood 332a for a target cell when using the multiple-multiplication model 318. The following briefly summarizes the multiplication operations 334a, 334b, and 334c in the order shown in FIG. 3B, although any order could be used. First, the accelerated genotype-imputation system 106 performs the multiplication operation 334a by multiplying a transition constant coefficient 322 (e.g., P0) for a column representing a target marker variant and summed adjacent-marker intermediate allele likelihoods 324 (e.g., Sum[m−1]) for an adjacent marker variant. In some cases, the summed adjacent-marker intermediate allele likelihoods 324 is normalized (e.g., Norm[m−1]*Sum[m−1]). For brevity, this disclosure uses an apostrophe as a shorthand to indicate normalized values (e.g., Sum′[m−1]).

Second, the accelerated genotype-imputation system 106 performs the multiplication operation 334b by multiplying a transition linear coefficient 326 (e.g., P1) for a column representing the target marker variant and an adjacent-marker intermediate allele likelihood 328a (e.g., A[m−1][k]) for the adjacent marker variant. In some cases, the adjacent-marker intermediate allele likelihood 328a is normalized (e.g., Norm[m−1]*A[m−1][k]). As further shown in FIG. 3B, the accelerated genotype-imputation system 106 performs a summing operation 340a by summing (i) a product of the transition constant coefficient 322 (P0) and the summed adjacent-marker intermediate allele likelihoods 324 (e.g., Norm[m−1]*Sum[m−1]) and (i) a product of the transition linear coefficient 326 (P0) and the adjacent-marker intermediate allele likelihood 328a (e.g., Norm[m−1]*A[m−1][k]).

Third, the accelerated genotype-imputation system 106 performs the multiplication operation 334c by multiplying an allele-likelihood factor 330a (e.g., Q0 or Q1) for the column representing the target marker variant and the summed product. As suggested above, allele-likelihood factor 330a may constitute an allele-likelihood factor (e.g., Q0) corresponding to a sample reference haplotype allele of a target haplotype represented by a row or another allele-likelihood factor (e.g., Q1) corresponding to a sample alternate haplotype of the target haplotype. Based on multiplying the allele-likelihood factor 330a (e.g., Q0 or Q1) and the summed product (P1[m]*Norm[m−1]*A[m−1][k]+P0[m]*Norm[m−1]*Sum[m−1]), the accelerated genotype-imputation system 106 determines the intermediate allele likelihood 332a (e.g., A[m][k]) using the multiple-multiplication model 318.

When using the multiple-multiplication model 318, in some embodiments, the accelerated genotype-imputation system 106 determines an intermediate allele likelihood for a target cell during both an alpha pass and a beta pass. The values corresponding to the adjacent marker variant (m−1) accordingly differ for a target cell from an alpha pass to a beta pass. Indeed, by using the multiple-multiplication model 318, the accelerated genotype-imputation system 106 determines one value for a column representing the target marker variant by performing the multiplication operation 334a for an alpha pass and another value for the column representing the target marker variant by performing the multiplication operation 334a for a beta pass. Further, by using the multiple-multiplication model 318, the accelerated genotype-imputation system 106 determines one value per row and per column by performing the multiplication operation 334b for an alpha pass and another value per row and per column by performing the multiplication operation 334b for a beta pass.

In contrast to the multiple-multiplication model 318, the accelerated genotype-imputation system 106 performs a multiplication operation 334d as part of determining an intermediate allele likelihood 332b for the target cell when using the single-multiplication model 320. As an overview, the accelerated genotype-imputation system 106 performs the multiplication operation 334d by multiplying a first transition-aware allele-likelihood factor 338 and an adjacent-marker intermediate allele likelihood 328b. By further performing a summing operation 340b to sum an adjacent-marker-transition-factor-aware allele likelihood 342 and a summed-adjacent-marker transition-aware allele-likelihood factor 336, the accelerated genotype-imputation system 106 determines the intermediate allele likelihood 332b for the target cell.

By using the single-multiplication model 320 shown in FIG. 3B, in some embodiments, the accelerated genotype-imputation system 106 selects a haplotype allele 330b for a column representing the target variant marker within a haplotype matrix. In some cases, the haplotype allele 330b takes the form of an S bit that selects a value representing a haplotype allele to pass to a downstream logic. For instance, in certain embodiments, the accelerated genotype-imputation system 106 selects the haplotype allele 330b by identifying either (i) an allele-likelihood factor (e.g., Q0) corresponding to a sample reference haplotype allele of a target haplotype represented by a row or (ii) another allele-likelihood factor (e.g., Q1) corresponding to a sample alternate haplotype of the target haplotype. Based on the identified allele-likelihood factor (e.g., Q0 or Q1), the accelerated genotype-imputation system 106 passes or sends a corresponding value representing a haplotype allele downstream for used in the summed-adjacent-marker transition-aware allele-likelihood factor 336 and the first transition-aware allele-likelihood factor 338. Indeed, as further shown in FIG. 3B, the accelerated genotype-imputation system 106 uses the selected haplotype allele 330b as part of the summed-adjacent-marker transition-aware allele-likelihood factor 336 and the first transition-aware allele-likelihood factor 338 as part of the single-multiplication model 320.

As suggested above, in some embodiments, the accelerated genotype-imputation system 106 predetermines the first transition-aware allele-likelihood factor 338 and a second transition-aware allele-likelihood factor (the latter as part of the summed-adjacent-marker transition-aware allele-likelihood factor 336) before determining intermediate allele likelihoods for a column representing a target marker variant within a haplotype matrix. To predetermine the first transition-aware allele-likelihood factor 338, in some embodiments, the accelerated genotype-imputation system 106 multiplies an allele-likelihood factor (e.g., Q[m][Allele]) corresponding to a particular type of haplotype allele for the haplotype allele 330b and a transition constant coefficient (P0) for transitioning between haplotypes from the haplotype reference panel. To predetermine the summed-adjacent-marker transition-aware allele-likelihood factor 336, the accelerated genotype-imputation system 106 multiplies the allele-likelihood factor (e.g., Q[m][Allele]), a transition linear coefficient (e.g., P1) for transitioning between haplotypes from the haplotype reference panel, and summed adjacent-marker intermediate allele likelihoods 324 (e.g., Sum′[m−1]) for an adjacent marker variant.

During a pass of a haplotype matrix, the accelerated genotype-imputation system 106 also determines the adjacent-marker intermediate allele likelihood 328b for an adjacent cell representing an adjacent variant marker and the target haplotype. Indeed, in some embodiments, as the accelerated genotype-imputation system 106 performs a pass of determining intermediate allele likelihoods column by column of a haplotype matrix, the accelerated genotype-imputation system 106 determines the adjacent-marker intermediate allele likelihood 328b for the adjacent cell before reaching the target cell.

Having predetermined the first transition-aware allele-likelihood factor 338—and the adjacent-marker intermediate allele likelihood 328b—the accelerated genotype-imputation system 106 can perform a single, pass-concurrent multiplication operation for the target cell. In particular, as shown in FIG. 3B, the accelerated genotype-imputation system 106 performs the multiplication operation 334d by multiplying the first transition-aware allele-likelihood factor 338 (e.g., Q[m][Allele]*P1 [m]) and the adjacent-marker intermediate allele likelihood 328b (e.g., A[m−1][k]). As an output of the multiplication operation 334d, the accelerated genotype-imputation system 106 generates the adjacent-marker-transition-factor-aware allele likelihood 342 (e.g., Q[m][Allele]*P1[m]*A′[m−1]).

As further shown in FIG. 3B, the accelerated genotype-imputation system 106 further determines the intermediate allele likelihood 332b for the target cell by performing the summing operation 340b. In particular, the accelerated genotype-imputation system sums the adjacent-marker-transition-factor-aware allele likelihood 342 (e.g., Q[m][Allele]*P1[m]*A′[m−1]) and the summed-adjacent-marker transition-aware allele-likelihood factor 336 (e.g., Q[m][Allele]*P0[m]*Sum′[m−1]) to determine the intermediate allele likelihood 332b (e.g., A[m][k]).

As suggested above, by performing the three multiplication operations 334a-334c for each target cell using the multiple-multiplication model 318, the accelerated genotype-imputation system 106 would perform 3,000 multiplication operations for each row representing a haplotype from a haplotype reference panel. By contrast, by performing the multiplication operation 334d for each target cell using the single-multiplication model 320, the accelerated genotype-imputation system 106 reduces processing to roughly 1,000 multiplication operations for each row representing a haplotype from a haplotype reference panel. Because multiplication operations on a configurable processor, such as an FPGA, consume considerable processing, the single-multiplication model 320 significantly reduces both time and computer processing to determine intermediate allele likelihoods and output allele likelihoods.

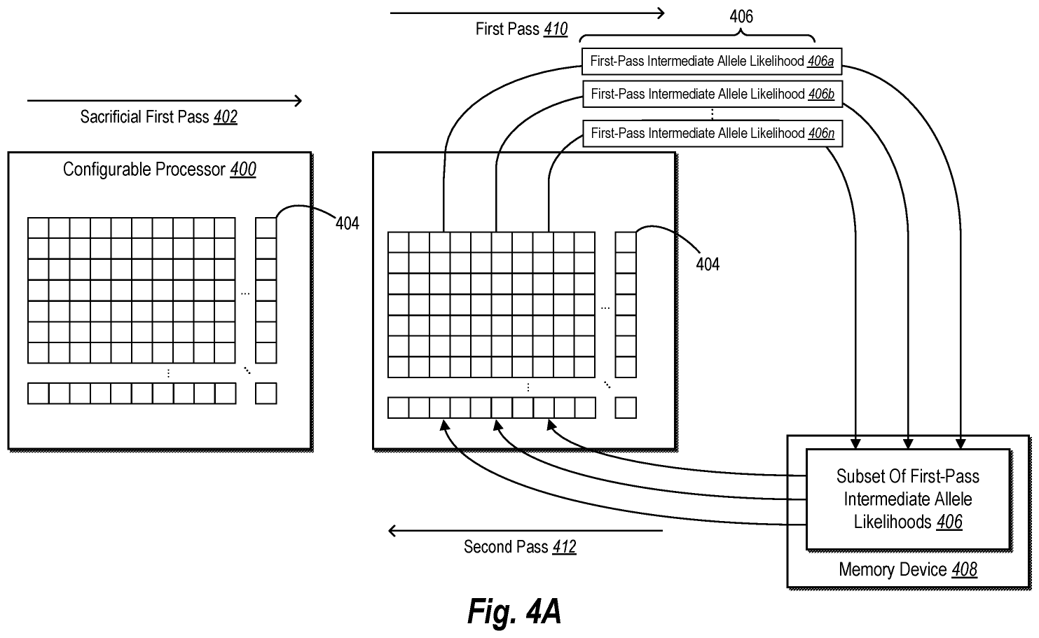

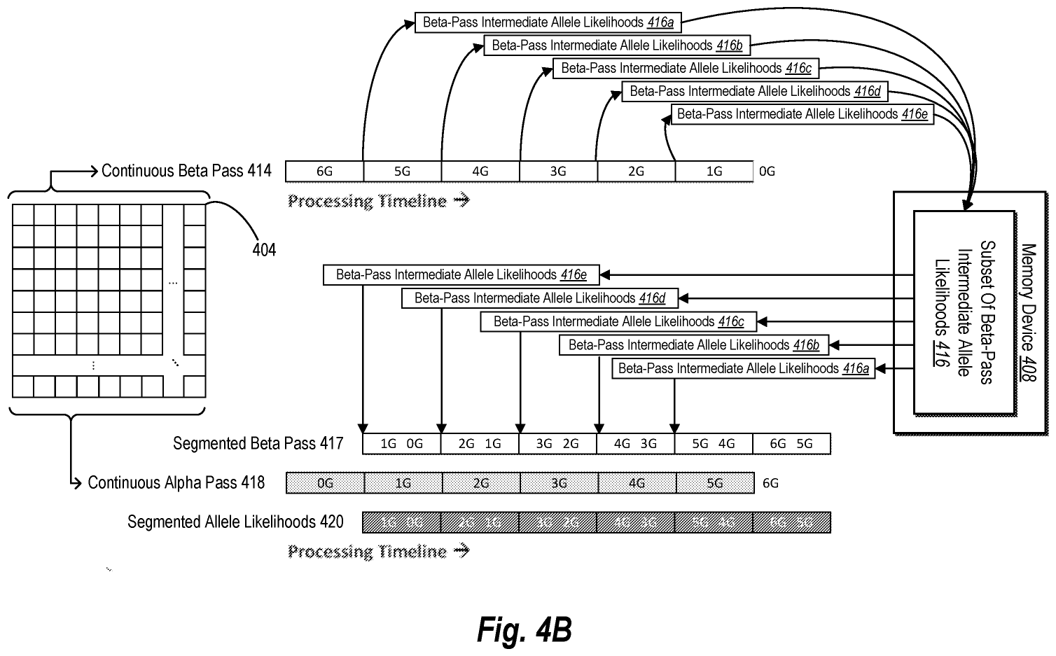

In addition or in the alternative to performing a single, pass-concurrent multiplication operation for a target cell, in some embodiments, the accelerated genotype-imputation system 106 can store and use intermediate-allele-likelihood subsets to hot start determining certain intermediate allele likelihoods during a pass across a haplotype matrix. In accordance with one or more embodiments, FIG. 4A depicts the accelerated genotype-imputation system 106 storing and accessing subsets of intermediate allele likelihoods corresponding to groups of marker variants to hot-start intermediate-allele-likelihood determinations during one or more passes across a haplotype matrix. FIG. 4B depicts the accelerated genotype-imputation system 106 (i) determining and storing subsets of intermediate allele likelihoods corresponding to columns of marker variants that are grouped together and (ii) generating sets of intermediate allele likelihoods for passes across the haplotype matrix by using the intermediate-allele-likelihood subsets as hot-start points.

As shown in FIG. 4A, in some embodiments, the accelerated genotype-imputation system 106 uses a configurable processor 400 to perform a sacrificial first pass 402 of determining intermediate allele likelihoods across cells of a haplotype matrix 404. This disclosure refers to the sacrificial first pass 402 as “sacrificial” because the accelerated genotype-imputation system 106 performs the sacrificial first pass 402 for the purpose of determining a subset of first-pass intermediate allele likelihoods 406 corresponding to a subset of marker variants. Other than hot-start points for regenerating first-pass intermediate allele likelihoods, in some embodiments, the accelerated genotype-imputation system 106 does not directly use the intermediate allele likelihoods determined during the sacrificial first pass 402.

When performing the sacrificial first pass 402, the accelerated genotype-imputation system 106 may perform a forward pass or reverse pass (or an alpha pass or a beta pass). As suggested above, in a forward pass, the accelerated genotype-imputation system 106 generates forward intermediate allele likelihoods of a genomic region comprising haplotype alleles. By contrast, in a reverse pass, the accelerated genotype-imputation system 106 generates reverse intermediate allele likelihoods of a genomic region comprising haplotype alleles. Because the accelerated genotype-imputation system 106 performs both a forward pass (e.g., a second pass) and a reverse pass (e.g., a first pass) as a basis for generating allele likelihoods—regardless of a sacrificial pass's direction—the direction of the sacrificial pass should not affect the allele likelihoods (e.g., R0, R1). Regardless of the direction, in some embodiments, the accelerated genotype-imputation system 106 performs the sacrificial first pass 402 by determining—cell by cell and column by column of the haplotype matrix 404—an intermediate allele likelihood for each cell representing a combination of marker variant and haplotype from a haplotype reference panel. By performing the sacrificial first pass 402, the accelerated genotype-imputation system 106 determines, utilizing the configurable processor 400, first-pass intermediate allele likelihoods of a genomic region from a genomic sample comprising haplotype alleles corresponding to a set of haplotypes given a set of marker variants.

After performing the sacrificial first pass 402, as further shown in FIG. 4A, the accelerated genotype-imputation system 106 identifies first-pass intermediate allele likelihoods 406a-406n from among the first-pass intermediate allele likelihoods determined from the sacrificial first pass 402. For instance, in some embodiments, the accelerated genotype-imputation system 106 identifies groups of marker variants—such as groups of 20, 100, 500, or 1,000 marker variants—and (ii) selects a first-pass intermediate allele likelihoods from each group of marker variants to include within the subset of first-pass intermediate allele likelihoods 406. Accordingly, in some embodiments, the accelerated genotype-imputation system 106 selects an intermediate allele likelihood for one column of marker variants for every 20, 100, 500, or 1,000 columns of marker variants within the haplotype matrix 404.

As shown in FIG. 4A, the first-pass intermediate allele likelihoods 406a-406n represent intermediate allele likelihoods from columns selected every threshold number of columns representing a group of marker variants. Together, the first-pass intermediate allele likelihoods 406a, 406b, and up through 406n constitute the subset of first-pass intermediate allele likelihoods 406.

In addition to identifying the subset of first-pass intermediate allele likelihoods 406, as further shown in FIG. 4A, the accelerated genotype-imputation system 106 stores the subset of first-pass intermediate allele likelihoods 406 on a memory device 408. As suggest above, the values in the haplotype matrix 404 after the sacrificial first pass 402 would saturate or prove too much to store in the on-chip memory of the configurable processor 400. To reduce and redistribute the voluminous data of the haplotype matrix 404 after the sacrificial first pass 402, the accelerated genotype-imputation system 106 stores the subset of first-pass intermediate allele likelihoods 406 on DRAM, SRAM, or other suitable memory for the memory device 408. The memory device 408 may be on chip with the configurable processor 400 or off chip from the configurable processor 400. Without saturating the memory of the configurable processor 400, the accelerated genotype-imputation system 106 can access the subset of first-pass intermediate allele likelihoods 406 from the memory device 408 as hot-start points for determining intermediate allele likelihoods in a first pass 410.

As further shown in FIG. 4A, in some embodiments, the accelerated genotype-imputation system 106 regenerates the first-pass intermediate allele likelihoods from the sacrificial first pass 402 by utilizing the subset of first-pass intermediate allele likelihoods 406 to initialize allele-likelihood determinations at the groups of marker variants. In particular, when performing the first pass 410, the accelerated genotype-imputation system 106 (i) uses one of the first-pass intermediate allele likelihoods 406a-406n as the intermediate allele likelihoods for one column of marker variants for every 20, 100, 500, or 1,000 columns of marker variants and (ii) uses one of the first-pass intermediate allele likelihoods 406a-406n as a hot-start point to determine subsequent intermediate allele likelihoods in subsequent columns during the first pass 410.

As further shown in FIG. 4A, the accelerated genotype-imputation system 106 can further perform a second pass 412 of determining second-pass intermediate allele likelihoods in a different direction from the first pass 410. In particular, the accelerated genotype-imputation system 106 determines, utilizing the configurable processor 400, second-pass intermediate allele likelihoods of a genomic region comprising haplotype alleles corresponding to the set of haplotypes given the set of marker variants. Based on the regenerated first-pass intermediate allele likelihoods and the second-pass intermediate allele likelihoods, the accelerated genotype-imputation system 106 generates allele likelihoods of the genomic region comprising the haplotype alleles.

FIG. 4B depicts a more detailed embodiment of the accelerated genotype-imputation system 106 using intermediate-allele-likelihood subsets as hot-start points. As shown in FIG. 4B, the accelerated genotype-imputation system 106 determines and stores a subset of beta-pass intermediate allele likelihoods 416 corresponding to respective columns of marker variants grouped in groups of marker variants, including marker-variant groups 1G-6G. The accelerated genotype-imputation system 106 subsequently accesses the subset of beta-pass intermediate allele likelihoods 416 and uses the individually stored intermediate allele likelihoods as hot-start points to generate intermediate allele likelihoods in both an alpha pass and a beta pass across the haplotype matrix 404.

As shown in FIG. 4B, as a sacrificial beta pass, the accelerated genotype-imputation system 106 performs a continuous beta pass 414 of determining beta-pass intermediate allele likelihoods corresponding to a set of haplotypes and a set of marker variants represented by the haplotype matrix 404. In particular, the accelerated genotype-imputation system 106 performs the continuous beta pass 414 by determining a beta-pass intermediate allele likelihood for each cell within the haplotype matrix 404. While FIG. 4B uses the continuous beta pass 414 as an example sacrificial pass, the accelerated genotype-imputation system 106 can likewise use a continuous alpha pass as the sacrificial pass. Due to space constraints, however, FIG. 4B depicts the continuous beta pass 414 in a horizontal block. But the continuous beta pass 414 generates beta-pass intermediate allele likelihoods (also known as beta values) for each cell and each column of cells within the haplotype matrix 404. Although the continuous beta pass 414 is generally performed in a reverse direction (and typically represented from right to left) across the haplotype matrix 404, FIG. 4B depicts groups 6G-1G of columns representing groups of marker variants in reverse numerical order along a horizontal processing timeline.

After performing the continuous beta pass 414, the accelerated genotype-imputation system 106 identifies and stores, within the memory device 408, beta-pass intermediate allele likelihoods 416a-416e as the subset of beta-pass intermediate allele likelihoods 416. As indicated by FIG. 4B, each of the beta-pass intermediate allele likelihoods 416a-416e correspond to a column representing a marker variant from a group of columns (e.g., one of groups 1G-5G). For example, the beta-pass intermediate allele likelihoods 416a represents a column of intermediate allele likelihood values selected from a group 5G of columns representing a group of marker variants. By contrast, the beta-pass intermediate allele likelihoods 416b represents a column of intermediate allele likelihood values selected from a group 4G of columns representing a group of marker variants. The beta-pass intermediate allele likelihoods 416c, 416d, and 416e each likewise represent a column of intermediate allele likelihood values selected from one of a group 3G, 2G, and 1G of columns, respectively, representing different groups of marker variants. In some cases, the accelerated genotype-imputation system 106 selects a last column of intermediate allele likelihoods (e.g., beta-pass intermediate allele likelihoods 416e) as the beta-pass intermediate allele likelihoods to store for a particular group of columns/marker variants (e.g., 1G).

After storing the beta-pass intermediate allele likelihoods 416a-416e as the subset of beta-pass intermediate allele likelihoods 416 in the memory device 408, as further shown in FIG. 4B, the accelerated genotype-imputation system 106 performs a segmented beta pass 417. When performing the segmented beta pass 417, the accelerated genotype-imputation system 106 regenerates the intermediate allele likelihood values determined in the continuous beta pass 414. To conserve memory on a chip for a configurable processor or other processor, however, the accelerated genotype-imputation system 106 loads beta-pass intermediate allele likelihoods from the subset of beta-pass intermediate allele likelihoods 416 at certain columns to initialize (or hot start) determining beta-pass intermediate allele likelihoods for an adjacent column—without having to redetermine the subset of beta-pass intermediate allele likelihoods 416 during the segmented beta pass 417.

As further depicted in FIG. 4B, the accelerated genotype-imputation system 106 loads the relevant stored subset of beta-pass intermediate allele likelihoods at the relevant column during the segmented beta pass 417. Although the segmented beta pass 417 is generally performed in a reverse direction (and typically represented from right to left) across the haplotype matrix 404, FIG. 4B depicts groups of columns representing groups of marker variants proceeding in reverse numerical order along the horizontal processing timeline. As suggested above, if the accelerated genotype-imputation system 106 were to perform a sacrificial alpha pass instead of or in addition to a sacrificial beta pass, the accelerated genotype-imputation system 106 would likewise perform a segmented alpha pass.

To illustrate the sequence of the segmented beta pass 417, in some embodiments, the accelerated genotype-imputation system 106 determines beta-pass intermediate allele likelihoods for the initial group 0G of columns and subsequently loads the beta-pass intermediate allele likelihoods 416e for the first column of the first group 1G of columns. Based on the beta-pass intermediate allele likelihoods 416e, the accelerated genotype-imputation system 106 determines the beta-pass intermediate allele likelihoods of a column adjacent to the first column within the first group 1G of columns. Similarly, the accelerated genotype-imputation system 106 determines beta-pass intermediate allele likelihoods for the entire first group 1G of columns and subsequently loads the beta-pass intermediate allele likelihoods 416d for the first column of the second group 2G of columns. Based on the beta-pass intermediate allele likelihoods 416d, the accelerated genotype-imputation system 106 determines the beta-pass intermediate allele likelihoods of a column adjacent to the first column within the second group 2G of columns.

In addition to the segmented beta pass 417, as further shown in FIG. 4B, the accelerated genotype-imputation system 106 also performs a continuous alpha pass 418 of determining alpha-pass intermediate allele likelihoods corresponding to the set of haplotypes and the set of marker variants represented by the haplotype matrix 404. In particular, the accelerated genotype-imputation system 106 performs the continuous alpha pass 418 by determining an alpha-pass intermediate allele likelihood for each cell within the haplotype matrix 404. Because the continuous alpha pass 418 is generally performed in a forward direction (and typically represented from left to right) across the haplotype matrix 404, FIG. 4B depicts the groups 0G-6G of columns representing groups of marker variants in numerical order along a horizontal processing timeline.

As further shown in FIG. 4B, as both the segmented beta pass 417 and the continuous alpha pass 418 progress, the accelerated genotype-imputation system 106 determines segmented allele likelihoods 420. To illustrate the sequence of the segmented allele likelihoods 420, in some embodiments, the accelerated genotype-imputation system 106 determines allele likelihoods for the initial group 0G of columns by multiplying sums of corresponding beta-pass and alpha-pass intermediate allele likelihoods. When the accelerated genotype-imputation system 106 subsequently loads the beta-pass intermediate allele likelihoods 416e for the first column of the first group 1G of columns and determines the alpha-pass intermediate allele likelihood for the first column as part of the continuous alpha pass 418, the accelerated genotype-imputation system 106 multiplies the respective sums of the beta-pass intermediate allele likelihoods 416e and alpha-pass intermediate allele likelihoods for the first column of the first group 1G of columns. Based on such a multiplication of sums, the accelerated genotype-imputation system 106 determines the allele likelihoods (R0 and R1) for the first column of the first group 1G of columns. In some embodiments, the accelerated genotype-imputation system 106 overwrites the respective sums of the beta-pass intermediate allele likelihoods 416e and alpha-pass intermediate allele likelihoods for the first column of the first group 1G of columns with the allele likelihoods for the first column of the first group 1G of columns.

As a further illustration, in some embodiments, the accelerated genotype-imputation system 106 determines allele likelihoods for the first group 1G of columns by multiplying sums of corresponding beta-pass and alpha-pass intermediate allele likelihoods. When the accelerated genotype-imputation system 106 loads the beta-pass intermediate allele likelihoods 416d for the first column of the second group 2G of columns and determines the alpha-pass intermediate allele likelihood for the first column as part of the continuous alpha pass 418, the accelerated genotype-imputation system 106 multiplies the respective sums of the beta-pass intermediate allele likelihoods 416d and alpha-pass intermediate allele likelihoods for the first column of the second group 2G of columns. Based on such a multiplication of sums, the accelerated genotype-imputation system 106 determines the allele likelihoods (R0 and R1) for the first column of the second group 2G of columns and (in some cases) overwrites the respective sums with the allele likelihoods for the first column of the second group 2G of columns.

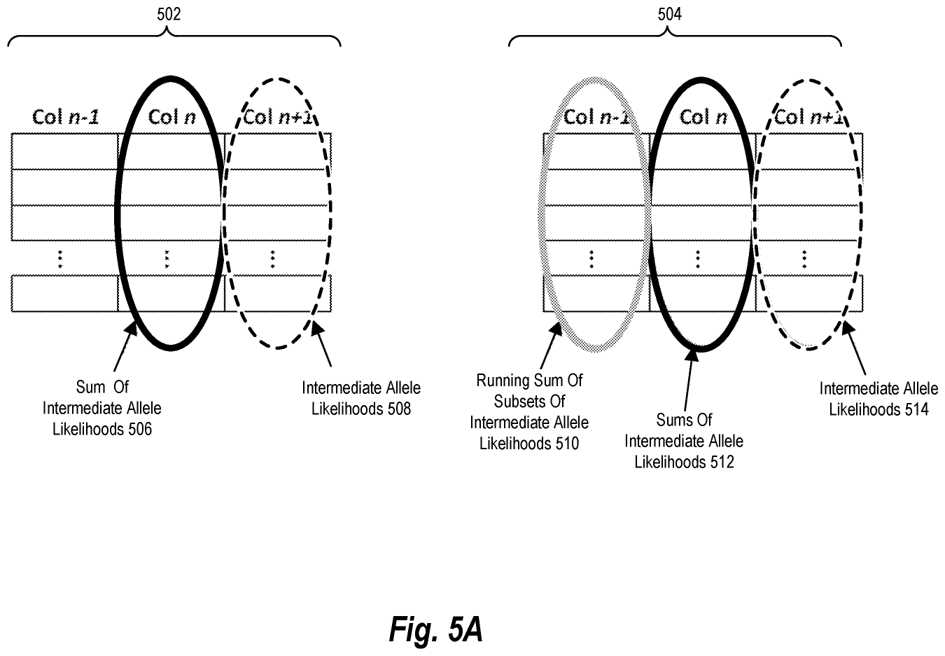

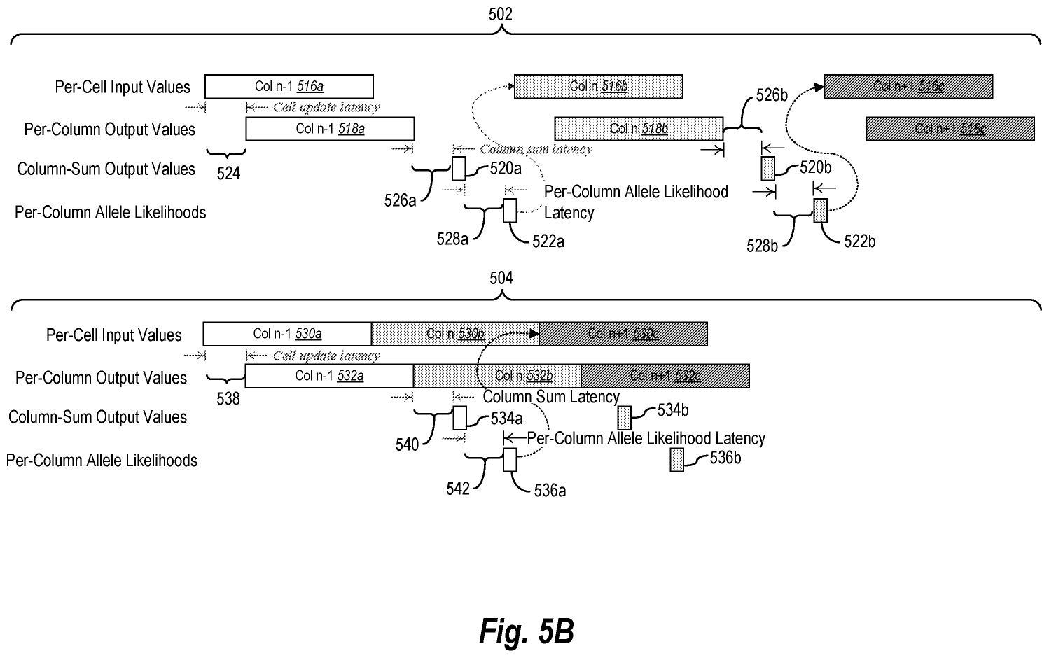

In addition or in the alternative to using intermediate-allele-likelihood subsets as hot-start points, in some embodiments, the accelerated genotype-imputation system 106 determines and uses running sums of intermediate allele likelihoods to expedite performing a pass of determining intermediate allele likelihoods across a haplotype matrix. In accordance with one or more embodiments, FIG. 5A depicts the accelerated genotype-imputation system 106 determining a running sum of intermediate allele likelihoods of a genomic region exhibiting haplotype alleles for one or more haplotypes in a column n−1 (representing a first marker variant) as running inputs for determining individual intermediate allele likelihoods of the genomic region exhibiting the haplotype alleles in a column n (representing a second marker variant). FIG. 5B depicts a comparison of the accelerated genotype-imputation system 106 using a full sum model and a running sum model to determine column sums of intermediate likelihoods and the effect of such models on latency periods.

As shown in FIG. 5A, the accelerated genotype-imputation system 106 performs a full-column-sum model 502 to determine intermediate allele likelihoods for columns representing different variant markers. When performing the full-column-sum model 502, for instance, the accelerated genotype-imputation system 106 determines a sum of intermediate allele likelihoods 506 for column n representing the second marker variant before determining intermediate allele likelihoods 508 for column n+1 representing a third marker variant. When performing the full-column-sum model 502, the full-column-sum model 502 causes a processor to wait latency periods for determining of intermediate allele likelihoods 506 for column n—and generating allele likelihoods for column n—before beginning to determine intermediate allele likelihoods 508 for column n+1. Because a haplotype matrix can require determining values for the equivalent of millions, billions, or trillions of cells and determining intermediate allele likelihoods for cells in parallel is more efficient than a serial approach, such latency periods prove costly and significantly slow down a process that can average around 17.5 hours to phase and impute haplotype allele likelihoods for a single marker allele corresponding to a genomic region.

In contrast to the full-column-sum model 502, in some embodiments, the accelerated genotype-imputation system 106 performs a running-column-sum model 504 to determine intermediate allele likelihoods for columns representing different variant markers. As shown in FIG. 5A, for instance, the accelerated genotype-imputation system 106 determines running sums of intermediate allele likelihoods 510 of a genomic region exhibiting haplotype alleles for one or more haplotypes given the first marker variant represented by column n−1. By using the running sums of intermediate allele likelihoods 510 as running inputs for column n, the accelerated genotype-imputation system 106 determines sums of intermediate allele likelihoods 512 of the genomic region exhibiting the haplotype alleles given the second marker variant represented by column n.

When performing the running-column-sum model 504, the accelerated genotype-imputation system 106 expedites determining intermediate allele likelihoods for haplotype-matrix cells in parallel. As further shown in FIG. 5A, the accelerated genotype-imputation system 106 further determines such running sums of intermediate allele likelihoods given the second marker variant represented by column n. By using the running sums of intermediate allele likelihoods for column n as running inputs, the accelerated genotype-imputation system 106 similarly determines intermediate allele likelihoods 514 of the genomic region exhibiting the haplotype alleles given the third marker variant represented by column n+1. Indeed, the accelerated genotype-imputation system 106 can derive (or otherwise determine) the intermediate allele likelihoods 514 of column n+1 based on the sums of intermediate allele likelihoods 512 of column n. Unlike the full-column-sum model 502, by using the running-column-sum model 504, the accelerated genotype-imputation system 106 does not need to wait to determine sums of intermediate allele likelihoods for one column before determining individual (or sums of) allele likelihoods for another column within a haplotype matrix.

FIG. 5B depicts a comparison of the accelerated genotype-imputation system 106 performing the full-column-sum model 502 and the running-column-sum model 504 in further detail with relative timing of per-column input values and output values. When performing the full-column-sum model 502, the accelerated genotype-imputation system 106 can determine an intermediate allele likelihood (e.g., A[m][k]) for a target cell by performing the multiplication operations 334a, 334b, and 334c and the summing operation 340a depicted in FIG. 3B and described above. Indeed, this disclosure calls the full-column-sum model 502 a “full column sum” because one such multiplication operation requires summing an entire column of intermediate allele likelihoods (e.g., A[m][k] values). In particular, when performing the multiplication operation 334a depicted in FIG. 3B, in some embodiments, the accelerated genotype-imputation system 106 multiplies a transition constant coefficient (P0) for a column representing a target marker variant and normalized summed adjacent-marker intermediate allele likelihoods (Sum′[m−1]) for an adjacent marker variant represented by a column. Because the summed adjacent-marker intermediate allele likelihoods (Sum′[m−1]) requires summing intermediate allele likelihoods for an entire column representing an adjacent marker variant within a haplotype matrix, the full-column-sum model 502 imposes latency periods depicted in FIG. 5B on a processor to determine and sum intermediate allele likelihoods for a column representing a target marker variant—without performing other parallel operations.

As shown in FIG. 5B, when performing the full-column-sum model 502, the accelerated genotype-imputation system 106 inputs per-cell column input values 516a into cells of column n−1 to determine per-cell column output values 518a for column n−1. As suggested above, in some embodiments of the full-column-sum model 502, the per-cell column input values 516a comprise allele-likelihood factors (Q0 or Q1), transition coefficients (P1[m] and P0[m]), summed adjacent-marker intermediate allele likelihoods (Sum′[m−1]), and normalization values (Norm[m−1]) for each cell in column n−1. Based on the per-cell column input values 516a, in some embodiments, the accelerated genotype-imputation system 106 determines the per-cell column output values 518a in the form of intermediate allele likelihoods represented as alpha values (e.g., A[m][k] values) or beta values (e.g., B[m][k] values) for an alpha pass or a beta pass, respectively. FIG. 5B depicts the time to determine the per-cell column output values 518a from the cells of column n−1 as a cell update latency 524.

Based on the per-cell column output values 518a, as part of the full-column-sum model 502, the accelerated genotype-imputation system 106 determines column sum output values 520a for column n−1. For instance, in some embodiments, the accelerated genotype-imputation system 106 determines a sum of alpha values (Sum[m]=Σₖ₌₀^{k-1}A[m][k]) for column n−1 and a sum of beta values (Sum[m]=Σₖ₌₀^{k-1}B[m][k]) for column n−1. Based on the column sum output values 520a, the accelerated genotype-imputation system 106 determines per-column allele likelihoods 522a for column n−1. For example, the accelerated genotype-imputation system 106 multiplies a sum of alpha values (Sum[m]=Σₖ₌₀^{k-1}A[m][k]) for column n−1 and a sum of beta values (Sum[m]=Σₖ₌₀^{k-1}B[m][k]) to determine allele likelihoods (R0 and R1) for column n−1.

When performing the full-column-sum model 502, as shown in FIG. 5B, the accelerated genotype-imputation system 106 determines the column sum output values 520a and the per-column allele likelihoods 522a before inputting per-column input values 516b into cells of column n to determine per-cell column output values 518b for column n. Because a processor of the accelerated genotype-imputation system 106 determines the column sum output values 520a and the per-column allele likelihoods 522a before inputting per-column input values 516b, the full-column-sum model 502 creates a column sum latency 526a and a per-column allele-likelihood latency 528a depicted in FIG. 5B. In other words, the full-column-sum model 502 requires a processor to wait through a latency period for both of summing adjacent-marker intermediate allele likelihoods and generating allele likelihoods without performing other parallel operations for adjacent haplotype-matrix columns.

As further shown in FIG. 5B, the full-column-sum model 502 similarly creates a column sum latency and a per-column allele-likelihood latency between column n and column n+1. The accelerated genotype-imputation system 106 determines column sum output values 520b and per-column allele likelihoods 522b before inputting per-column input values 516c into cells of column n+1 to determine per-cell column output values 518c for column n+1. Because a processor of the accelerated genotype-imputation system 106 determines the column sum output values 520b and the per-column allele likelihoods 522b before inputting per-column input values 516c, as with other haplotype-matrix columns, the full-column-sum model 502 likewise creates a column sum latency 526b and a per-column allele-likelihood latency 528b.

In contrast to the full-column-sum model 502, the accelerated genotype-imputation system 106 eliminates such empty latency periods in performing the running-column-sum model 504. For example, in some embodiments, the accelerated genotype-imputation system 106 determines, for column n−1 representing an adjacent marker variant, a running sum of a first subset of intermediate allele likelihoods (e.g., Σ_{k(S==0)}A[m−1][k]) of the genomic region comprising a first type of haplotype allele from one or more haplotypes. Similarly, the accelerated genotype-imputation system 106 determines, for column n−1 representing the adjacent marker variant, a running sum of a second subset of intermediate allele likelihoods (e.g., Σ_{k(S==1)}A[m−1][k]) of the genomic region comprising a second type of haplotype allele from the one or more haplotypes. As indicated above, in some cases, the first type of haplotype allele comprises a sample reference haplotype allele (e.g., S[k][m] value is 0), and the second type of haplotype allele comprises a sample alternate haplotype allele (e.g., S[k][m] value is 1).

Based on the running sum of the first subset of intermediate allele likelihoods and the running sum of the second subset of intermediate allele likelihoods, the accelerated genotype-imputation system 106 determines, for a column n representing a target marker variant, sums of intermediate allele likelihoods (e.g., Sum[m]) of the genomic region comprising haplotype alleles from haplotypes of the haplotype reference panel. For instance, in some embodiments, the accelerated genotype-imputation system 106 determines a sum of intermediate allele likelihoods from an alpha pass and a sum of intermediate allele likelihoods from a beta pass. Based on the sums of intermediate allele likelihoods, the accelerated genotype-imputation system 106 generates, for column n representing the target marker variant, allele likelihoods (R0 and R1) of the genomic region comprising the haplotype alleles.

As noted above, the accelerated genotype-imputation system 106 can predetermine certain variables before a pass of a haplotype matrix to expedite the pass. In some cases, for instance, the accelerated genotype-imputation system 106 predetermines and accounts for various per-cell column input values as part of the running-column-sum model 504. For example, in some embodiments, the accelerated genotype-imputation system 106 predetermines a first transition-aware allele-likelihood factor corresponding to rows for the first type of haplotype allele (e.g., Q0[m]*P0[m]*(K−S₁)) and a second transition-aware allele-likelihood factor corresponding to rows for the second type of haplotype allele (e.g., Q1[m]*P0[m]*S₁). Accordingly, in addition to running sums, the accelerated genotype-imputation system 106 can determine a sum of intermediate allele likelihoods (e.g., Sum[m]) based further on the first transition-aware allele-likelihood factor corresponding to the rows for the first type of haplotype allele and the second transition-aware allele-likelihood factor corresponding to rows for the second type of haplotype allele.

As a further example, and as indicated above, the accelerated genotype-imputation system 106 can estimate adjacent-marker sums of intermediate allele likelihoods (e.g., Sum[m−1]) instead of summing all adjacent-marker intermediate allele likelihoods (e.g., A[m]1][k] values) to determine adjacent-marker sums of intermediate allele likelihoods (e.g., Sum[m−1]). Accordingly, in some embodiments, the accelerated genotype-imputation system 106 determines, for column n−1 representing the adjacent marker variant, adjacent-marker sums of intermediate allele likelihoods (e.g., Sum[m−1]) of the genomic region comprising the haplotype alleles based on the running sum of a first subset of intermediate allele likelihoods (e.g., Σ_{k(S==0)}A[m−1][k]) and the running sum of a second subset of intermediate allele likelihoods (e.g., Σ_{k(S==1)}A[m−1][k]).

According, in some embodiments, the accelerated genotype-imputation system 106 determines, for column n representing the marker variant, the sums of intermediate allele likelihoods (Sum[m]) based on a combination of (i) the adjacent-marker sum of intermediate allele likelihoods, (ii) the first transition-aware allele-likelihood factor corresponding to the rows for the first type of haplotype allele, (iii) the running sum of a first subset of intermediate allele likelihoods, (iv) the running sum of a second subset of intermediate allele likelihoods, and (v) the second transition-aware allele-likelihood factor corresponding to rows for the second type of haplotype allele. In some such cases, for instance, the accelerated genotype-imputation system 106 determines a product of the adjacent-marker sums of intermediate allele likelihoods (Sum[m−1]) and the first transition-aware allele-likelihood factor corresponding to rows for the first type of haplotype allele (Q0[m]*P0[m]*(K−S₁)) and adds the product to the second transition-aware allele-likelihood factor corresponding to rows for the second type of haplotype allele (Q1[m]*P0[m]*S₁).

In addition to determining running sums, in some cases, the accelerated genotype-imputation system 106 multiplies the running sums of subsets of intermediate allele likelihoods by transition-aware allele-likelihood factors as part of determining a sum of intermediate allele likelihoods (Sum[m]) for column n representing the target marker variant. For instance, in some embodiments, the accelerated genotype-imputation system 106 (i) multiplies the running sum of the first subset of intermediate allele likelihoods by a first transition-aware allele-likelihood factor (e.g., Q0[m]*P1[m]*Σ_{k(S==0)}A[m−1][k]) and multiplies the running sum of the second subset of intermediate allele likelihoods by a second transition-aware allele-likelihood factor (e.g., Q1[m]*P1[m]*Σ_{k(S==1)}A[m−1][k]. Based on the multiplied running sum of the first subset of intermediate allele likelihoods and the multiplied running sum of the second subset of intermediate allele likelihoods, the accelerated genotype-imputation system 106 determines, for the column n representing the target marker variant, a sum of intermediate allele likelihoods (Sum[m]).

According, in some embodiments, the accelerated genotype-imputation system 106 determines, for column n representing the marker variant, a sum of intermediate allele likelihoods (Sum[m]) by summing (a) the multiplied running sum of the first subset of intermediate allele likelihoods, (b) the multiplied running sum of the second subset of intermediate allele likelihoods, and (c) a product of (i) a normalization value for the adjacent marker variant, (ii) a product of an adjacent-marker sum of intermediate allele likelihoods, and (iii) a sum of the first transition-aware allele-likelihood factor corresponding to rows for the first type of haplotype allele and the second transition-aware allele-likelihood factor corresponding to rows for the second type of haplotype allele.

FIG. 5B illustrates the effect of the running-column-sum model 504 on various latency periods. As shown in FIG. 5B, when performing the running-column-sum model 504, the accelerated genotype-imputation system 106 inputs per-cell column input values 530b into cells of column n to determine per-cell column output values 532b from the cells of column n. As suggested above, in some embodiments of the running-column-sum model 504, the per-cell column input values 530b for each cell in column n comprise (i) a normalization value for an adjacent marker variant (e.g., Norm[m−1]), (ii) estimated adjacent-marker sums of intermediate allele likelihoods (e.g., Sum[m−1]), (iii) a first transition-aware allele-likelihood factor corresponding to rows for the first type of haplotype allele (e.g., Q0[m]*P0[m]*(K−S₁)), (iv) a second transition-aware allele-likelihood factor corresponding to rows for the second type of haplotype allele (e.g., Q1[m]*P0[m]*S₁), (v) a multiplied running sum of a first subset of intermediate allele likelihoods (e.g., Q0[m]*P1[m]*Σ_{k(S==0)}A[m−1][k]), and (v) a multiplied running sum of a second subset of intermediate allele likelihoods (e.g., Q1[m]*P1[m]*Σ_{k(S==1)}A[m−1][k]). Based on the per-cell column input values 530b, in some embodiments, the accelerated genotype-imputation system 106 determines the per-cell column output values 532b in the form of intermediate allele likelihoods represented as alpha values (e.g., A[m][k] values) or beta values (e.g., B[m][k] values) for an alpha pass or a beta pass, respectively.

Because of the running-column-sum model 504, as shown in FIG. 5B, the accelerated genotype-imputation system 106 determines column sum output values 534b for column n in the form of sums of intermediate allele likelihoods (e.g., Sum[m]) before finishing a determination of every intermediate allele likelihood in the per-cell column output values 532b. Indeed, as further indicated by FIG. 5B, the accelerated genotype-imputation system 106 determines the column sum output values 534b while also determining column sum output values 534a for column n−1 and per-column allele likelihoods 536a for column n−1. Accordingly, the accelerated genotype-imputation system 106 inputs per-cell column input values 530b and determines column sum output values 534b during both (i) a column sum latency 540 for column n−1 and (ii) a per-column allele-likelihood latency 542 for column n−1. The running-column-sum model 504 accordingly ensures that that a processor of the accelerated genotype-imputation system 106 determines intermediate allele likelihoods for column n during (rather than wait through) the column sum latency 540 for column n−1 and the per-column allele-likelihood latency 542 for column n−1.

As further indicated by FIG. 5B, in some embodiments, the accelerated genotype-imputation system 106 applies the running-column-sum model 504 to column n−1 and column n+1. For instance, the accelerated genotype-imputation system 106 inputs per-cell column input values 530c for column n+1 and determines column sum output values 532c for column n+1 while also determining column sum output values 534b for column n and per-column allele likelihoods 536b for column n—thereby ensuring that a processor the accelerated genotype-imputation system 106 does not wait through column sum latency and per-column allele-likelihood latency for column n without performing parallel operations for other columns.

Although not depicted in FIG. 5B, in some embodiments, the accelerated genotype-imputation system 106 can use running sums of intermediate allele likelihoods from column n−2 to determine a sum of intermediate allele likelihoods for column n−1. In particular, the accelerated genotype-imputation system 106 inputs per-cell column input values 530a for column n−1 and determines column sum output values 534a for column n−1 while also determining column sum output values for column n−2 and per-column allele likelihoods for column n−2. Accordingly, while FIG. 5B depicts a cell update latency 538 representing the time and processing to determine the per-cell column output values 532a from the cells of column n−1, in some cases, the accelerated genotype-imputation system 106 uses a processor of the accelerated genotype-imputation system 106 to determine other values for a haplotype matrix during the cell update latency 538.

As noted above, in some embodiments, the accelerated genotype-imputation system 106 intelligently transfers data to increase throughput on a configurable processor or other processor. In accordance with one or more embodiments, FIG. 6 illustrates the accelerated genotype-imputation system 106 storing haplotype-allele-indicator data for a haplotype matrix on a memory device and accessing the stored haplotype-allele indicator data to determine values as part of a pass across a haplotype matrix.

As noted above, HMM-based genotype imputations can require determining and storing enormous amounts of data, such as values for millions, billions, or trillions of cells in a haplotype matrix. For example, in some embodiments, the accelerated genotype-imputation system 106 inputs values representing haplotype alleles into each cell of a haplotype matrix, such as (i) one “S” bit indicating a sample reference haplotype allele of a particular haplotype and (ii) another “S” bit indicating a sample alternate haplotype allele of the particular haplotype. As noted above, this disclosure refers to such input values representing haplotype alleles as haplotype-allele-indicator data for a haplotype matrix. Because haplotype-allele-indicator data for a haplotype matrix with millions, billions, or trillions of cells can consume more multiple gigabytes of memory, haplotype-allele-indicator data taxes the bandwidth of high-speed buses for configurable processors, such as a Peripheral Component Interconnect Express (PCIe), or other interfaces that connect processor cards with other hardware within a computing device.

To save bandwidth on a PCIe or other interface, as shown in FIG. 6, the accelerated genotype-imputation system 106 stores, on a memory device 600, haplotype-allele-indicator data 602a for a haplotype matrix. In some cases, the accelerated genotype-imputation system 106 stores the haplotype-allele-indicator data 602a on on-chip DRAM, SRAM, or other suitable memory. Because the haplotype-allele-indicator data 602a is readily accessible, the accelerated genotype-imputation system 106 can access and transfer the haplotype-allele-indicator data 602a from the memory device 600 to a configurable processor 604 to perform a pass of determining intermediate allele likelihoods across a haplotype matrix. For instance, in some embodiments, the accelerated genotype-imputation system 106 uses the configurable processor 604 to access, from the memory device 600, the haplotype-allele-indicator data 602a for the haplotype matrix to generate allele likelihoods for a genotype imputation model.

Because the haplotype-allele-indicator data 602a (or “S” bit data) is the same format for haploid or diploid genotype imputation, the accelerated genotype-imputation system 106 can store and access the haplotype-allele-indicator data 602a for a hidden Markov haploid or diploid genotype imputation model. Accordingly, the accelerated genotype-imputation system 106 can use the configurable processor 604 to access, from the memory device 600, the haplotype-allele-indicator data 602a for the haplotype matrix to generate allele likelihoods utilizing either a hidden Markov haploid genotype imputation model or a hidden Markov diploid genotype imputation model. When input to a haplotype matrix for a pass, FIG. 6 depicts the input data as haplotype-allele-indicator data 602b on a matrix under analysis by the configurable processor 604.

To perform approximately 40,000 HMM-computation tasks for a single processor thread at approximately 60 seconds, in some embodiments, the accelerated genotype-imputation system 106 requires approximately 10 gigabytes per second of PCIe throughput with a margin of 6 gigabytes per second available during a pass. By storing on and accessing from on-chip DRAM (or other on-chip memory) the haplotype-allele-indicator data 602a for a haplotype matrix, in some embodiments, the accelerated genotype-imputation system 106 saves 4 or more gigabytes per second of PCIe bandwidth.

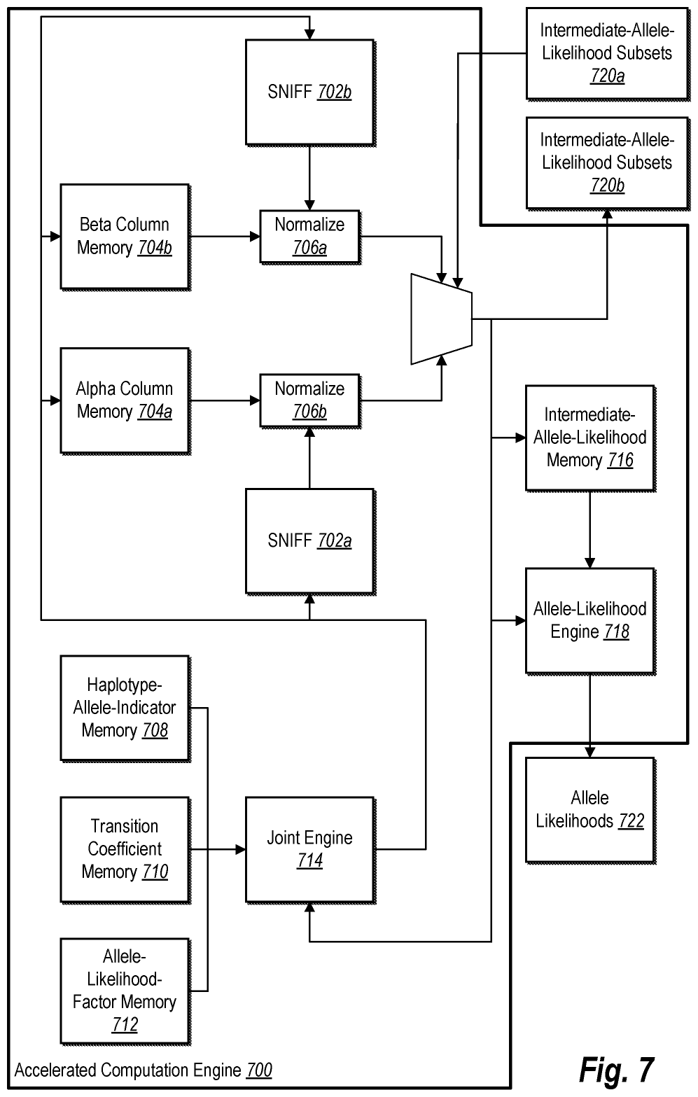

As noted above, in some embodiments, the accelerated genotype-imputation system 106 includes and uses customized architecture to run a genotype imputation model, such as GLIMPSE. In accordance with one or more embodiments, FIG. 7 illustrates an accelerated computation engine 700 comprising various customized engines and memory devices to determine allele likelihoods 722 using a genotype imputation model. The following paragraphs describe the various memory devices and operations used to determine the allele likelihoods 722. While the accelerated computation engine 700 depicted in FIG. 7 represents memory devices and engines for a haploid HMM computation, a similar accelerated computation engine may be used for a diploid HMM computation.

As shown in FIG. 7, for example, the accelerated computation engine 700 includes an alpha column memory 704a and a beta column memory 704b. In some embodiments, the alpha column memory 704a and the beta column memory 704b store pre-normalized intermediate allele likelihoods for an alpha pass and a beta pass, respectively. In particular, in certain implementations, the alpha column memory 704a and the beta column memory 704b store one column of pre-normalized alpha values (e.g., A[m][k] values) and one column of pre-normalized beta values (e.g., B[m][k] values), respectively. In terms of format, the alpha column memory 704a and the beta column memory 704b can each store values organized by K×Z_{ABwide} bits—that is, K number of rows representing haplotypes in Z width of bits for stored pre-normalized alpha values or beta values.

As further shown in FIG. 7, the accelerated computation engine 700 includes a haplotype-allele-indicator memory 708. The haplotype-allele-indicator memory 708 stores haplotype-allele-indicator data (or “S” bit data) comprising inputs values representing haplotype alleles for each cell of a haplotype matrix. This disclosure describes haplotype-allele-indicator data above with respect to FIGS. 2B and 6. In terms of format, the haplotype-allele-indicator memory 708 can store values or bits of haplotype-allele-indicator data organized as M×K bits—that is, M number of columns representing marker variants and K number of rows representing haplotypes from a haplotype reference panel. As indicated above, in some embodiments, the accelerated genotype-imputation system 106 transfers haplotype-allele-indicator data from on-chip DRAM or another memory device to the haplotype-allele-indicator memory 708 to perform a pass of a haplotype matrix.

In addition to the haplotype-allele-indicator memory 708, the accelerated computation engine 700 includes a transition coefficient memory 710. The transition coefficient memory 710 stores transition coefficients (e.g., P0 and P1 values) corresponding to columns or cells of a haplotype matrix. The transition coefficient memory 710 can store values for transition coefficients organized as 2×M×Zₚ bits—that is, two sections or blocks of values (e.g., one section for P0 values and one section for P1 values) in M number of columns representing marker variants in Zₚ bit width of inputted P0 and P1 values.

In addition to the transition coefficient memory 710, the accelerated computation engine 700 includes an allele-likelihood-factor memory 712. The allele-likelihood-factor memory 712 stores allele-likelihood factors (e.g., Q0 and Q1 values) corresponding to columns or cells of a haplotype matrix. The allele-likelihood-factor memory 712 can store values for allele-likelihood factors organized as 2×M×Z_{Q} bits—that is, two sections or blocks of values (e.g., one section for Q0 values and one section for Q1 values) in M number of columns representing marker variants in Z_{Q} bit width of inputted Q0 and Q1 values.

As further shown in FIG. 7, the accelerated computation engine 700 also includes an intermediate-allele-likelihood memory 716. The intermediate-allele-likelihood memory 716 stores intermediate allele likelihoods for a haplotype matrix. For instance, in some cases, the intermediate-allele-likelihood memory 716 stores alpha values and beta values determined across a full haplotype matrix. In terms of organization, the intermediate-allele-likelihood memory 716 can store intermediate allele likelihoods organized as W×K×Z_{AB} bits—that is, W number of columns in a marker-variant group, K number of rows representing haplotypes, and Z width of bits for stored normalized alpha values or beta values. Accordingly, in some embodiments, the intermediate-allele-likelihood memory 716 organizes alpha values or beta values by groups of marker variants to be compatible with subsets of pass intermediate allele likelihoods that initialize determining intermediate allele likelihoods at hot-start points.

By using the customized architecture of the accelerated computation engine 700, in some embodiments, the accelerated genotype-imputation system 106 determines the allele likelihoods 722 for one or more of a cell, column, or haplotype matrix. As shown in FIG. 7, for instance, the accelerated computation engine 700 uses a SNIFF 702a to generate an alpha normalization value and a SNIFF 702a to generate a beta normalization value. The accelerated computation engine 700 further applies the normalization value(s) from the SNIFF 702a to normalize 706a adjacent-marker intermediate likelihoods values from a column of alpha values stored in the alpha column memory 704a. Similarly, the accelerated computation engine 700 applies the normalization value(s) from the SNIFF 702b to normalize 706b adjacent-marker intermediate likelihoods values from a column of beta values stored in the beta column memory 704b.

As further shown in FIG. 7, the accelerated computation engine 700 uses a joint engine 714 to determine intermediate likelihoods values for target cells with a haplotype matrix. In particular, the accelerated computation engine 700 (i) receives normalized adjacent-marker intermediate allele likelihoods indirectly from the alpha column memory 704a and the beta column memory 704b and (ii) combines haplotype-allele indicators from the haplotype-allele-indicator memory 708, transition coefficients from the transition coefficient memory 710, and allele-likelihood factors from the allele-likelihood-factor memory 712 with the normalized adjacent-marker intermediate allele likelihoods to determine (iii) intermediate allele likelihoods for target cells stored in the intermediate-allele-likelihood memory 716. The accelerated computation engine 700 further uses an allele-likelihood engine 718 to determine the allele likelihoods 722 for target cells based on the intermediate allele likelihoods stored in the intermediate-allele-likelihood memory 716.

As further indicated in FIG. 7, in some embodiments, the accelerated computation engine 700 receives, from a memory device, intermediate-allele-likelihood subsets 720a corresponding to marker-variant groups. Consistent with the disclosure above, in some cases, the accelerated computation engine 700 uses the intermediate-allele-likelihood subsets to initialize allele-likelihood determinations at groups of marker variants—thereby regenerating first-pass intermediate allele likelihoods. As further indicated, the accelerated computation engine 700 also performs a sacrificial first pass and determines intermediate-allele-likelihood subsets 720b corresponding to marker-variant groups that can be stored on the memory device and later accessed to initialize allele-likelihood determinations at corresponding groups of marker variants.

Indeed, in some cases, the accelerated genotype-imputation system 106 can use the accelerated computation engine 700 to determine and access intermediate-allele-likelihood subsets as described above with respect to FIGS. 4A-4B. Further, in some embodiments, the accelerated genotype-imputation system 106 uses the accelerated computation engine 700 to determine single, pass-concurrent multiplication operations, determine and use running sums of subsets of intermediate allele likelihoods, or execute other embodiments described above with respect to FIGS. 3A-3B and 5A-5B.

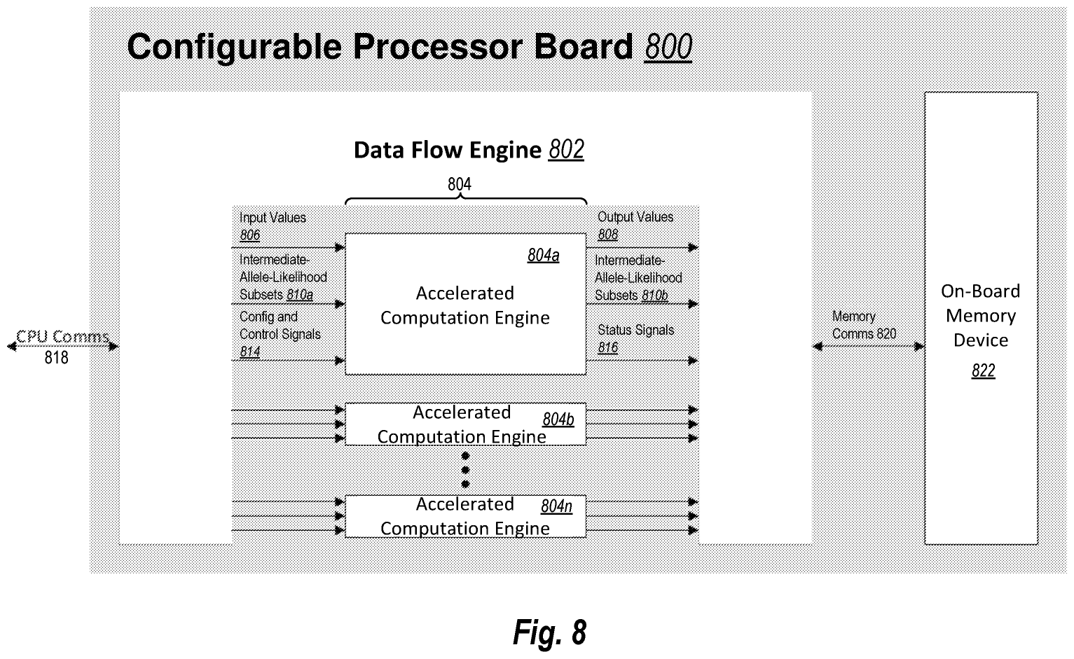

In addition to an accelerated computation engine as part of a customized architecture, in some embodiments, the accelerated genotype-imputation system 106 includes a data flow engine that can que and distribute HMM-computation tasks to an accelerated computation engine in a cluster of accelerated computation engines and manage data communications with a central processing unit (CPU), memory, and accelerated computation engines. In accordance with one or more embodiments, FIG. 8 depicts a configurable processor board 800 comprising a data flow engine 802, a cluster of accelerated computation engines 804, and an on-board memory device 822 for performing a genotype imputation model. As depicted in FIG. 8, the data flow engine 802 interacts and interfaces with the cluster of accelerated computation engines 804, the on-board memory device 822, and a CPU to que, distribute, or otherwise manage data for HMM-computation tasks. While the following paragraphs describe interactions and data exchanges between the data flow engine 802 and an accelerated computation engine 804a from the cluster of accelerated computation engines 804, the same interactions and data exchanges can be performed by the data flow engine 802 with each of accelerated computation engines 804b-804n.

As indicated by FIG. 8, for example, configurable processor board 800 is part of a local server device (e.g., the local device 110 shown in FIG. 1) or part of a sequencing device (e.g., the sequencing device 102 shown in FIG. 1). As part of such a computing device, in some embodiments, the data flow engine 802 on the configurable processor board 800 includes an PCIe interface for an FPGA and a Double Data Rate (DDR) interface to interface with the on-board memory device 822, such as DRAM.

In addition to or as part of functioning as an interface, in some embodiments, the data flow engine 802 sends and receives data to and from the CPU, the on-board memory device 822, and other hardware on the accelerated genotype-imputation system 106 for determining intermediate allele likelihoods, allele likelihoods, or other HMM computations. As part of CPU communications 818, in some embodiments, the data flow engine 802 receives a data indicator from the CPU to perform genotype imputation for one or more genomic regions of genomic samples based on prior genotype likelihoods derived from nucleotide-fragment reads. As part of memory communications 820, in some cases, the data flow engine 802 sends and receives input or output requests with the on-board memory device 822 to store or access data for genotype imputation or phasing. Such requests may include, for instance, sending or receiving a column of intermediate allele likelihoods (e.g., one column of alpha values or beta values) or intermediate-allele-likelihood subsets as hot-start points.

As just indicated, in some embodiments, the accelerated genotype-imputation system 106 can exchange intermediate-allele-likelihood subsets as hot-start points between the data flow engine 802 and the on-board memory device 822. For instance, in some cases, the accelerated genotype-imputation system 106 (i) sends, from the on-board memory device 822 to the data flow engine 802, a subset of first-pass intermediate allele likelihoods and (ii) sends, from the data flow engine 802 to an accelerated computation engine 804a of the cluster of accelerated computation engines 804, the subset of first-pass intermediate allele likelihoods to regenerate first-pass intermediate allele likelihoods based on the subset of first-pass intermediate allele likelihoods.

In addition to the CPU communications 818 and the memory communications 820, in some embodiments, the data flow engine 802 distributes HMM-computation tasks to individual accelerated computation engines from the cluster of accelerated computation engines 804. To illustrate, in some cases, the data flow engine assigns a single HMM-computation task—to a single accelerated computation engine from the cluster of accelerated computation engines 804—for a haplotype matrix of approximately 50 million cells resulting in approximately 40,000 haplotype calls. While other HMM-computation tasks may be bigger or smaller than the foregoing example, in some embodiments, each of the individual HMM-computations tasks includes inputs and output values for such a haplotype matrix.

As indicated in FIG. 8, for instance, the data flow engine 802 can send input values 806 to the accelerated computation engine 804a for a target column or haplotype matrix for genotype imputation or receive output values 808 from the accelerated computation engine 804a for the target column or haplotype matrix, such as allele likelihoods or intermediate-allele-likelihood subsets. The data flow engine 802 can likewise (i) receive intermediate-allele-likelihood subsets 810b as hot-start points from a sacrificial first pass of the accelerated computation engine 804a or (ii) send intermediate-allele-likelihood subsets 810a as hot-start points to the accelerated computation engine 804a to regenerate a column of intermediate allele likelihoods initially determined in a sacrificial first pass.

As an example of the input values 806 and the output values 808, in some embodiments, the accelerated genotype-imputation system 106 sends, from the data flow engine 802 to respective accelerated computation engines of the cluster of accelerated computation engines 804, respective sets of input values comprising allele-likelihood factors, transition coefficients, and haplotype-allele values. Based on the respective sets of input values, the respective accelerated computation engines determine respective sets of intermediate allele likelihoods corresponding to respective subsets of marker variants and subsets of haplotypes.

To further illustrate, in certain implementations, the accelerated genotype-imputation system 106 (i) sends, from the data flow engine 802 to the accelerated computation engine 804a, a first set of input values comprising allele-likelihood factors, transition coefficients, and haplotype-allele values and (ii) sends, from the data flow engine 802 to the accelerated computation engine 804b, a second set of input values comprising allele-likelihood factors, transition coefficients, and haplotype-allele values. Based on the first set of input values, the accelerated computation engine 804a determines a first set of intermediate allele likelihoods corresponding to a first subset of marker variants and a first subset of haplotypes. Similarly, based on the second set of input values, the accelerated computation engine 804b determines a second set of intermediate allele likelihoods corresponding to a second subset of marker variants and a second subset of haplotypes.

As an example of the intermediate-allele-likelihood subsets 810a and 810b, in some embodiments, the accelerated genotype-imputation system 106 sends, from the data flow engine 802 to the accelerated computation engine 804a, a subset of first-pass intermediate allele likelihoods for the accelerated computation engine 804a to regenerate a first-pass intermediate allele likelihoods from a sacrificial pass. Similarly, the accelerated genotype-imputation system 106 sends, from the data flow engine 802 to the accelerated computation engine 804b, an additional subset of first-pass intermediate allele likelihoods for the accelerated computation engine 804b to regenerate additional first-pass intermediate allele likelihoods from an additional sacrificial pass.

In addition to distributing specific data for HMM-computation tasks, as further shown in FIG. 8, the data flow engine 802 ques HMM-computation tasks for individual accelerated computation engines from the cluster of accelerated computation engines 804 and performs further data exchanges with the on-board memory device 822. As shown in FIG. 8, for instance, the data flow engine 802 sends configuration-and-control signals 814 to the accelerated computation engine 804a, such as data indicators for a timing and order of HMM-computation tasks queued up for the accelerated computation engine 804a. Similarly, in some embodiments, the data flow engine 802 receives status signals 816 from the accelerated computation engine 804a concerning a status or completion of a particular HMM-computation task. Based on the status signals 816 from the accelerated computation engine 804a, the data flow engine 802 queues up additional HMM-computation tasks for the accelerated computation engine 804a or reorganizes or reorders other HMM-computation tasks for the accelerated computation engines 804b-804n. As part of such HMM-computation tasks, in some embodiments, the data flow engine 802 also receives and responds to DDR input or output requests from the on-board memory device 822.

As noted above, in some embodiments, the accelerated genotype-imputation system 106 can perform approximately 40,000 HMM-computation tasks in approximately 60 seconds, thereby expediting processing time by 600 times. The configurable processor board 800 depicted in FIG. 8 can be implemented to facilitate such speeds. If the accelerated genotype-imputation system 106 determines 1× alpha values and 2× beta values across a haplotype matrix of 2 trillion cells, the accelerated genotype-imputation system 106 must determine the equivalent of values for 6 trillion cells. Given 16 accelerated computation engines, the customized architecture in the configurable processor board 800 can perform approximately 40,000 HMM-computation tasks in approximately 60 seconds.

To illustrate, if “L” represents a level of parallelism for a given accelerated computation engine—to compute “L” alpha values and beta values per clock cycle—and a given accelerated computation engine has a core clock speed of 400 mHZ, a single accelerated computation engine can compute L cells/cycle×400M cycles per second in 60 seconds, which is the equivalent of L×24 billion alpha or beta cells. To compute values for 6 trillion cells at 24 billion cells per single accelerated computation engine, the L (or level of parallelism) would need to equal 16. Accordingly a set of 16 accelerated computation engines using the architecture in the configurable processor board 800 in FIG. 8 could perform approximately 40,000 HMM-computation tasks in approximately 60 seconds.

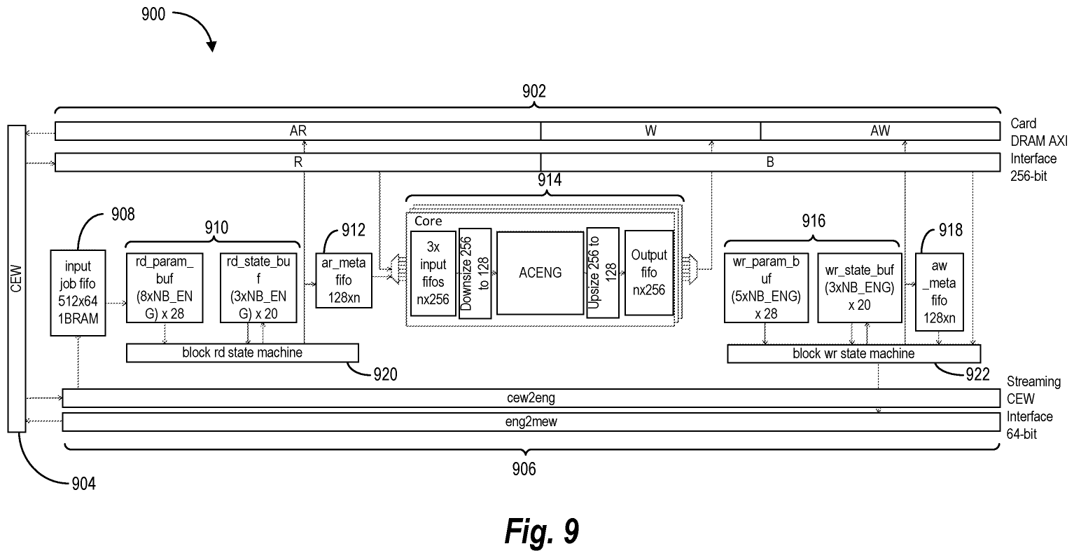

In some embodiments, an accelerated computation engine can be part of a larger hardware structure. In accordance with one or more embodiments, FIG. 9 depicts a schematic diagram 900 of an accelerated computation engine core 914 with surrounding interfaces and other hardware.

As shown in FIG. 9, the accelerated computation engine core 914 includes input first-in-first-outs (FIFOS) to receive data from a card DRAM advanced extensible interface (AXI) interface 902 and from an address read meta FIFO 912. The accelerated computation engine core 914 also includes an output FIFO to output HMM-computation values to a write channel of the card DRAM AXI interface 902. As further depicted within the accelerated computation engine core 914 in FIG. 9, each of the input FIFOs and output FIFO include corresponding converters for downsizing and upsizing data, respectively.

On each side of the accelerated computation engine core 914, the schematic diagram 900 includes buffers 910 and buffers 916. As part of the buffers 910, a read parameter buffer and a read stat buffer send or receive data from a block read state machine 920. As further shown in FIG. 9, the read parameter buffer receives data from an input job FIFO 908. As part of the buffers 916, a write parameter buffer and a write state buffer send or receive data from a block write state machine 922. Additionally, an address write meta FIFO 918 sends and receives data to and from the block write state machine 922 and (in some cases) to and from an address write channel of the card DRAM AXI interface 902.

As further shown in FIG. 9, the card DRAM AXI interface 902 includes multiple different channels. In particular, the card DRAM AXI interface 902 includes an address read (AR) channel that receives data from the block read state machine 920, a write (W) channel that receives output values from the accelerated computation engine core 914, and an address write (AW) channel that receives data from the block write state machine 922. Further, the card DRAM AXI interface 902 includes a read (R) channel that receives data from a Common Engine Wrapper (CEW) 904 and a write response (B) channel to which response information is signaled for write transactions.

Finally, as further shown in FIG. 9, the CEW 904 provides access to the job control infrastructure (e.g., configuration and control signals from the data flow engine 802), the card DRAM AXI interface 902, and host memory (e.g., the on-board memory device 822). Accordingly, by using the CEW 904, the accelerated genotype-imputation system 106 can exchange data with the card DRAM AXI interface 902 and a streaming CEW interface 906. For example, the CEW 904 sends configuration and control signals to and from the accelerated computation engine core 914.

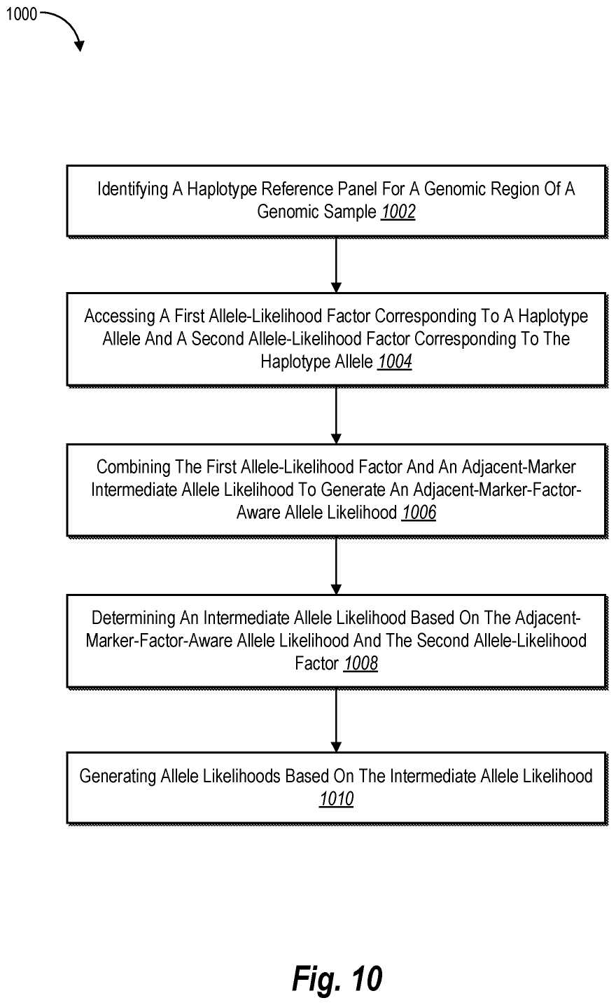

Turning now to FIG. 10, this figure illustrates a flowchart of a series of acts 1000 of determining an intermediate allele likelihood of a genomic region comprising a haplotype allele by running consolidated operations on a processor in accordance with one or more embodiments of the present disclosure. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer readable storage medium can comprise instructions that, when executed by one or more processors, cause a computing device or a system to perform the acts depicted in FIG. 10. In still further embodiments, a system comprising at least one processor and a non-transitory computer readable medium comprising instructions that, when executed by one or more processors, cause the system to perform the acts of FIG. 10.

As shown in FIG. 10, the acts 1000 include an act 1002 of identifying a haplotype reference panel for a genomic region of a genomic sample. In particular, in some embodiments, the act 1002 includes identifying, utilizing a genotype imputation model, a haplotype reference panel for a genomic region of a genomic sample. In some cases, the genotype imputation model comprises a hidden Markov genotype imputation model.

As further shown in FIG. 10, the acts 1000 include an act 1004 of accessing a first allele-likelihood factor corresponding to a haplotype allele and a second allele-likelihood factor corresponding to the haplotype allele. In particular, in some embodiments, the act 1004 includes accessing, from a memory device and for a marker variant, a first allele-likelihood factor corresponding to a haplotype allele from the haplotype reference panel and a second allele-likelihood factor corresponding to the haplotype allele. Relatedly, in some embodiments, the act 1004 includes accessing, from a memory device and for a marker variant, a first transition-aware allele-likelihood factor corresponding to a haplotype allele from the haplotype reference panel and a second transition-aware allele-likelihood factor corresponding to the haplotype allele. Further, in certain cases, the memory device comprises dynamic random-access memory (DRAM), dynamic random-access memory (SRAM), or a cache memory device.

For example, in some embodiments, accessing, from the memory device and for the marker variant, the first allele-likelihood factor and the second allele-likelihood factor comprises accessing, from the memory device and for the marker variant, a first transition-aware allele-likelihood factor corresponding to the haplotype allele from the haplotype reference panel and a second transition-aware allele-likelihood factor corresponding to the haplotype allele. In some cases, determining the first transition-aware allele-likelihood factor comprises combining an allele-likelihood factor and a transition linear coefficient. For instance, in certain implementations, the first allele-likelihood factor comprises an allele-likelihood factor for a sample reference haplotype allele or for a sample alternate haplotype allele; and the second allele-likelihood factor comprises the allele-likelihood factor for the sample reference haplotype allele or for the sample alternate haplotype allele.

Relatedly, in some embodiments, the acts 1000 further comprise predetermining the first transition-aware allele-likelihood factor and the second transition-aware allele-likelihood factor before determining one or more intermediate allele likelihoods corresponding to the marker variant as part of a pass across a haplotype matrix. Similarly, in some cases, the acts 1000 comprise predetermining the first transition-aware allele-likelihood factor and the second transition-aware allele-likelihood factor before determining one or more intermediate allele likelihoods corresponding to the marker variant. For instance, in some embodiments, the act 1004 includes predetermining the first transition-aware allele-likelihood factor by combining an allele-likelihood factor for the haplotype allele and a transition constant coefficient for transitioning between haplotypes from the haplotype reference panel; and predetermining the second transition-aware allele-likelihood factor by combining the allele-likelihood factor and a transition linear coefficient for transitioning between haplotypes from the haplotype reference panel.

As further shown in FIG. 10, the acts 1000 include an act 1006 of combining the first allele-likelihood factor and an adjacent-marker intermediate allele likelihood to generate an adjacent-marker-factor-aware allele likelihood. In particular, in certain implementations, the act 1006 includes combining the first allele-likelihood factor and an adjacent-marker intermediate allele likelihood of the genomic region comprising the haplotype allele given an adjacent marker variant to generate an adjacent-marker-factor-aware allele likelihood for the marker variant and a haplotype from the haplotype reference panel.

Further, in some cases, the act 1006 comprises combining, by a configurable processor, the first transition-aware allele-likelihood factor and an adjacent-marker intermediate allele likelihood of the genomic region comprising the haplotype allele given an adjacent marker variant to generate an adjacent-marker-transition-factor-aware allele likelihood for the marker variant and a haplotype from the haplotype reference panel. For instance, in some embodiments, the configurable processor comprises an application-specific integrated circuit (ASIC), an application-specific standard product (ASSP), a coarse-grained reconfigurable array (CGRA), or a field programmable gate array (FPGA).

To further illustrate, in some embodiments, combining the first allele-likelihood factor and the adjacent-marker intermediate allele likelihood comprises multiplying a first transition-aware allele-likelihood factor and the adjacent-marker intermediate allele likelihood without further multiplication operations to determine the intermediate allele likelihood. Relatedly, in certain implementations, combining the first transition-aware allele-likelihood factor and the adjacent-marker intermediate allele likelihood comprises multiplying the first transition-aware allele-likelihood factor and the adjacent-marker intermediate allele likelihood without further multiplication operations to determine the intermediate allele likelihood.

As further shown in FIG. 10, the acts 1000 include an act 1008 of determining an intermediate allele likelihood based on the adjacent-marker-factor-aware allele likelihood and the second allele-likelihood factor. In particular, in certain implementations, the act 1008 includes determining, for the marker variant and the haplotype, an intermediate allele likelihood of the genomic region comprising the haplotype allele based on the adjacent-marker-factor-aware allele likelihood and the second allele-likelihood factor. Further, in some cases, the act 1008 includes determining, by the configurable processor and for the marker variant and the haplotype, an intermediate allele likelihood of the genomic region comprising the haplotype allele based on the adjacent-marker-transition-factor-aware allele likelihood and the second transition-aware allele-likelihood factor.

Further, in some cases, determining the intermediate allele likelihood comprises determining the intermediate allele likelihood of the genomic region comprising a sample reference haplotype allele or a sample alternate haplotype allele. Relatedly, in certain cases, determining the intermediate allele likelihood based on the adjacent-marker-factor-aware allele likelihood and the second allele-likelihood factor comprises summing an adjacent-marker-transition-factor-aware allele likelihood and a summed-adjacent-marker transition-aware allele-likelihood factor.

As further shown in FIG. 10, the acts 1000 include an act 1010 of generating allele likelihoods based on the intermediate allele likelihood. In particular, in some implementations, the act 1008 comprises generating, for a set of marker variants corresponding to the genomic region, allele likelihoods of the genomic region comprising haplotype alleles from the haplotype reference panel based on the intermediate allele likelihood. Further, in some cases, the act 1010 includes generating, by the configurable processor and for a set of marker variants corresponding to the genomic region, allele likelihoods of the genomic region comprising haplotype alleles from the haplotype reference panel based on the intermediate allele likelihood.

In addition or in the alternative to the acts 1002-1010, in certain implementations, the acts 1000 further include sending, from the data flow engine to respective accelerated computation engines of a cluster of accelerated computation engines, respective sets of input values comprising allele-likelihood factors, transition coefficients, and haplotype-allele values; and determining, by the respective accelerated computation engines and based on the respective sets of input values, respective sets of intermediate allele likelihoods corresponding to respective subsets of marker variants and respective subsets of haplotypes. In some embodiments, the data flow engine corresponds to a cluster of accelerated computation engines.

To further illustrate, in some cases, the acts 1000 further include sending the respective sets of input values from the data flow engine to the respective accelerated computation engines by: sending, from the data flow engine to a first accelerated computation engine of the cluster of accelerated computation engines, a first set of input values comprising allele-likelihood factors, transition coefficients, and haplotype-allele values; sending, from the data flow engine to a second accelerated computation engine of the cluster of accelerated computation engines, a second set of input values comprising allele-likelihood factors, transition coefficients, and haplotype-allele values; and determining the respective sets of intermediate allele likelihoods by: determining, by the first accelerated computation engine and based on the first set of input values, a first set of intermediate allele likelihoods corresponding to a first subset of marker variants and a first subset of haplotypes; and determining, by the second accelerated computation engine and based on the second set of input values, a second set of intermediate allele likelihoods corresponding to a second subset of marker variants and a second subset of haplotypes.

As suggested above, in some cases, the acts 1000 further include accessing the second transition-aware allele-likelihood factor as part of a summed-adjacent-marker transition-aware allele-likelihood factor; and determining the intermediate allele likelihood based on the adjacent-marker-transition-factor-aware allele likelihood and the summed-adjacent-marker transition-aware allele-likelihood factor. Relatedly, in some implementations, the acts 1000 include predetermining the summed-adjacent-marker transition-aware allele-likelihood factor by combining an allele-likelihood factor for the haplotype allele, a transition constant coefficient for transitioning between haplotypes from the haplotype reference panel, and summed adjacent-marker intermediate allele likelihoods for the adjacent marker variant. As further suggested above, in some cases, the allele-likelihood factor for the haplotype allele comprises a reference allele-likelihood factor for a sample reference haplotype allele or an alternate allele-likelihood factor for a sample alternate haplotype allele.

Additionally, in certain implementations, the acts 1000 further include determining one or more nucleobase calls for the genomic region from the genomic sample based on the allele likelihoods of the genomic region and one or more variant nucleobase calls surrounding the genomic region.

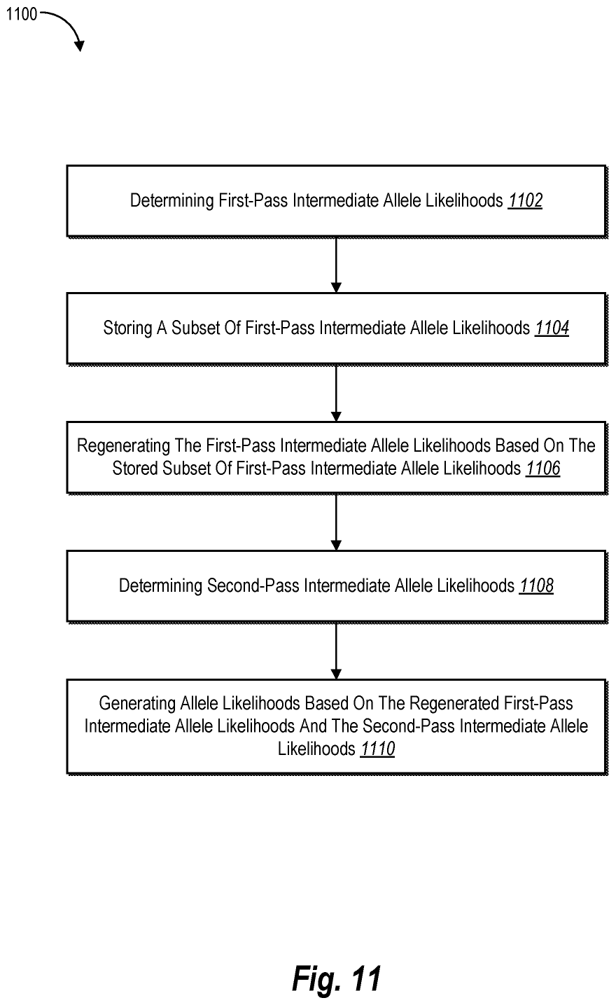

Turning now to FIG. 11, this figure illustrates a flowchart of a series of acts 1100 of determining and storing intermediate-allele-likelihood subsets as hot-start points corresponding to marker-variant groups and extemporaneously generating sets of intermediate allele likelihoods for a set of marker variants by using the intermediate-allele-likelihood subsets in accordance with one or more embodiments of the present disclosure. While FIG. 11 illustrates acts according to one embodiment, alternative embodiments may omit, add to, reorder, and/or modify any of the acts shown in FIG. 11. The acts of FIG. 11 can be performed as part of a method. Alternatively, a non-transitory computer readable storage medium can comprise instructions that, when executed by one or more processors, cause a computing device or a system to perform the acts depicted in FIG. 11. In still further embodiments, a system comprising at least one processor and a non-transitory computer readable medium comprising instructions that, when executed by one or more processors, cause the system to perform the acts of FIG. 11.

As shown in FIG. 11, the acts 1100 include an act 1102 of determining first-pass intermediate allele likelihoods. In particular, in some embodiments, the act 1102 includes determining, by performing a first pass, first-pass intermediate allele likelihoods of a genomic region from a genomic sample comprising haplotype alleles corresponding to a set of haplotypes given a set of marker variants. Further, in some cases, the act 1102 includes determining, utilizing a configurable processor performing a first pass, first-pass intermediate allele likelihoods of a genomic region from a genomic sample comprising haplotype alleles corresponding to a set of haplotypes given a set of marker variants. In certain cases, the configurable processor comprises an application-specific integrated circuit (ASIC), an application-specific standard product (ASSP), a coarse-grained reconfigurable array (CGRA), or a field programmable gate array (FPGA).

As further shown in FIG. 11, the acts 1100 include an act 1104 of storing a subset of first-pass intermediate allele likelihoods. In particular, in some embodiments, the act 1104 includes storing, on the memory device, a subset of first-pass intermediate allele likelihoods corresponding to a subset of marker variants for groups of marker variants. Further, in some cases, the act 1104 includes storing a subset of first-pass intermediate allele likelihoods corresponding to a subset of marker variants for groups of marker variants. In some cases, the memory device comprises dynamic random-access memory (DRAM), dynamic random-access memory (SRAM), or a cache memory device.

As further shown in FIG. 11, the acts 1100 include an act 1106 of regenerating the first-pass intermediate allele likelihoods based on the stored subset of first-pass intermediate allele likelihoods. In particular, in certain implementations, the act 1106 includes regenerating the first-pass intermediate allele likelihoods by utilizing the stored subset of first-pass intermediate allele likelihoods to initialize allele-likelihood determinations at the groups of marker variants. Further, in some embodiments, the act 1106 includes regenerating, utilizing the configurable processor, the first-pass intermediate allele likelihoods by utilizing the stored subset of first-pass intermediate allele likelihoods to initialize allele-likelihood determinations at the groups of marker variants.

Relatedly, in some cases, utilizing the stored subset of first-pass intermediate allele likelihoods to initialize allele-likelihood determinations at the groups of marker variants comprises: determining a first subset of first-pass intermediate allele likelihoods for a first group of marker variants based on a first stored column of first-pass intermediate allele likelihoods for an initial marker variant from the first group of marker variants; and determining a second subset of first-pass intermediate allele likelihoods for a second group of marker variants based on a second stored column of first-pass intermediate allele likelihoods for an initial marker variant from the second group of marker variants.

Relatedly, in some cases, the acts 1100 include storing the subset of first-pass intermediate allele likelihoods by storing, in dynamic random-access memory (DRAM), the subset of first-pass intermediate allele likelihoods; and utilizing the stored subset of first-pass intermediate allele likelihoods to initialize the allele-likelihood determinations at the groups of marker variants comprises accessing the stored subset of first-pass intermediate allele likelihoods from the DRAM.

As further shown in FIG. 11, the acts 1100 include an act 1108 of determining second-pass intermediate allele likelihoods. In particular, in certain implementations, the act 1108 includes determining, by performing a second pass, second-pass intermediate allele likelihoods of the genomic region comprising the haplotype alleles corresponding to the set of haplotypes given the set of marker variants. Further, in some cases, the act 1108 includes determining, utilizing the configurable processor performing a second pass, second-pass intermediate allele likelihoods of the genomic region comprising the haplotype alleles corresponding to the set of haplotypes given the set of marker variants.

As suggested above, in some cases, the acts 1100 include determining the first-pass intermediate allele likelihoods comprises determining, utilizing a reverse pass, reverse intermediate allele likelihoods of the genomic region comprising the haplotype alleles; and determining the second-pass intermediate allele likelihoods comprises determining, utilizing a forward pass, forward intermediate allele likelihoods of the genomic region comprising the haplotype alleles.

As further shown in FIG. 11, the acts 1100 include an act 1110 of generating allele likelihoods based on the regenerated first-pass intermediate allele likelihoods and the second-pass intermediate allele likelihoods. In particular, in certain implementations, the act 1110 includes generating allele likelihoods of the genomic region comprising the haplotype alleles based on the regenerated first-pass intermediate allele likelihoods and the second-pass intermediate allele likelihoods. Further, in some embodiments, the act 1110 includes generating, utilizing an output engine, allele likelihoods of the genomic region comprising the haplotype alleles based on the regenerated first-pass intermediate allele likelihoods and the second-pass intermediate allele likelihoods.

To illustrate, in some embodiments, generating the allele likelihoods based on the regenerated first-pass intermediate allele likelihoods and the second-pass intermediate allele likelihoods comprises: determining summed first-pass intermediate allele likelihoods for the set of marker variants based on the regenerated first-pass intermediate allele likelihoods; determining summed second-pass intermediate allele likelihoods for the set of marker variants based on the second-pass intermediate allele likelihoods; and determining the allele likelihoods based on the summed first-pass intermediate allele likelihoods and the summed second-pass intermediate allele likelihoods.

In addition or in the alternative to the acts 1102-1110, in certain implementations, the acts 1000 further include storing haplotype-allele-indicator data in a haplotype-allele-indicator memory; storing transition coefficients in a transition coefficient memory; and storing allele-likelihood factors in an allele-likelihood-factor memory. Further, in some embodiments, the acts 1000 include determining intermediate allele likelihood values using a joint engine.

As suggested above, in some cases, the acts 1100 further include sending, from a data flow engine to respective accelerated computation engines of a cluster of accelerated computation engines, respective sets of input values comprising allele-likelihood factors, transition coefficients, and haplotype-allele values; and determining, by the respective accelerated computation engines and based on the respective sets of input values, respective sets of intermediate allele likelihoods corresponding to respective subsets of marker variants and respective subsets of haplotypes. In some embodiments, the data flow engine corresponds to a cluster of accelerated computation engines.

To further illustrate, in some cases, the acts 1100 further include sending the respective sets of input values from the data flow engine to the respective accelerated computation engines by: sending, from the data flow engine to a first accelerated computation engine of the cluster of accelerated computation engines, a first set of input values comprising allele-likelihood factors, transition coefficients, and haplotype-allele values; sending, from the data flow engine to a second accelerated computation engine of the cluster of accelerated computation engines, a second set of input values comprising allele-likelihood factors, transition coefficients, and haplotype-allele values; and determining the respective sets of intermediate allele likelihoods by: determining, by the first accelerated computation engine and based on the first set of input values, a first set of intermediate allele likelihoods corresponding to a first subset of marker variants and a first subset of haplotypes; and determining, by the second accelerated computation engine and based on the second set of input values, a second set of intermediate allele likelihoods corresponding to a second subset of marker variants and a second subset of haplotypes.

As further suggested above, in some cases, the acts 1100 include sending, from the data flow engine to a first accelerated computation engine of the cluster of accelerated computation engines, the subset of first-pass intermediate allele likelihoods for the first accelerated computation engine to regenerate the first-pass intermediate allele likelihoods; and sending, from the data flow engine to a second accelerated computation engine from the cluster of accelerated computation engines, an additional subset of first-pass intermediate allele likelihoods for the second accelerated computation engine to regenerate additional first-pass intermediate allele likelihoods.

Further, in certain implementations, the acts 1100 include sending, from the memory device to the data flow engine, the subset of first-pass intermediate allele likelihoods; and sending, from the data flow engine to an accelerated computation engine, the subset of first-pass intermediate allele likelihoods to regenerate the first-pass intermediate allele likelihoods based on the subset of first-pass intermediate allele likelihoods. Additionally, in some cases, the acts 1100 includes storing, on the memory device, haplotype-allele-indicator data for a haplotype matrix; and accessing, from the memory device, the haplotype-allele-indicator data for the haplotype matrix to generate the allele likelihoods utilizing a hidden Markov haploid genotype imputation model or a hidden Markov diploid genotype imputation model.

As suggested above, in some cases, the acts 1100 include determining one or more nucleobase calls for the genomic region from the genomic sample based on the allele likelihoods of the genomic region and one or more variant nucleobase calls surrounding the genomic region.

As further suggested above, in certain implementations, the acts 1100 include storing, on dynamic random-access memory (DRAM), haplotype-allele-indicator data for a haplotype matrix; and accessing, by the configurable processor from the DRAM, the haplotype-allele-indicator data for the haplotype matrix to generate the allele likelihoods utilizing a hidden Markov haploid genotype imputation model or a hidden Markov diploid genotype imputation model.

Additionally or alternatively, in certain embodiments, the acts 1100 include determining, for an adjacent marker variant, a running sum of a first subset of intermediate allele likelihoods of the genomic region comprising a first type of haplotype allele from one or more haplotypes of the haplotype reference panel; determining, for the adjacent marker variant, a running sum of a second subset of intermediate allele likelihoods of the genomic region comprising a second type of haplotype allele from the one or more haplotypes; and determining, for the marker variant, sums of intermediate allele likelihoods of the genomic region comprising haplotype alleles from haplotypes of the haplotype reference panel based on the running sum of the first subset of intermediate allele likelihoods and the running sum of the second subset of intermediate allele likelihoods.

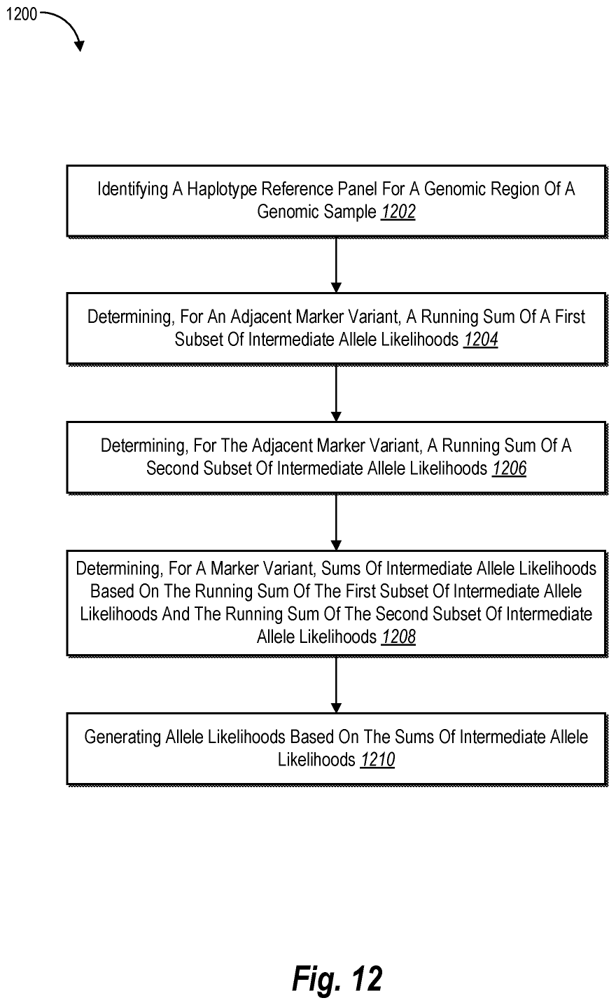

Turning now to FIG. 12, this figure illustrates a flowchart of a series of acts 1200 of determining running sums of intermediate allele likelihoods of a genomic region exhibiting haplotype alleles for one or more haplotypes given one marker variant and using the running sums as running inputs to determine individual intermediate allele likelihoods of the genomic region exhibiting the haplotype alleles given another marker variant in accordance with one or more embodiments of the present disclosure. While FIG. 12 illustrates acts according to one embodiment, alternative embodiments may omit, add to, reorder, and/or modify any of the acts shown in FIG. 12. The acts of FIG. 12 can be performed as part of a method. Alternatively, a non-transitory computer readable storage medium can comprise instructions that, when executed by one or more processors, cause a computing device or a system to perform the acts depicted in FIG. 12. In still further embodiments, a system comprising at least one processor and a non-transitory computer readable medium comprising instructions that, when executed by one or more processors, cause the system to perform the acts of FIG. 12.

As shown in FIG. 12, the acts 1200 include an act 1202 of identifying a haplotype reference panel for a genomic region of a genomic sample. In particular, in some embodiments, the act 1202 includes identifying, utilizing a genotype imputation model, a haplotype reference panel for a genomic region of a genomic sample.

As further shown in FIG. 12, the acts 1200 include an act 1204 of determining, for an adjacent marker variant, a running sum of a first subset of intermediate allele likelihoods. In particular, in some embodiments, the act 1204 includes determining, for an adjacent marker variant, a running sum of a first subset of intermediate allele likelihoods of the genomic region comprising a first type of haplotype allele from one or more haplotypes of the haplotype reference panel.

As further shown in FIG. 12, the acts 1200 include an act 1206 of determining, for the adjacent marker variant, a running sum of a second subset of intermediate allele likelihoods. In particular, in certain implementations, the act 1206 includes determining, for the adjacent marker variant, a running sum of a second subset of intermediate allele likelihoods of the genomic region comprising a second type of haplotype allele from the one or more haplotypes.

As noted above, in some embodiments, the first type of haplotype allele comprises a sample reference haplotype allele, and the second type of haplotype allele comprises a sample alternate haplotype allele.

As further shown in FIG. 12, the acts 1200 include an act 1208 of determining, for a marker variant, sums of intermediate allele likelihoods based on the running sum of the first subset of intermediate allele likelihoods and the running sum of the second subset of intermediate allele likelihoods. In particular, in certain implementations, the act 1208 includes determining, for a marker variant, sums of intermediate allele likelihoods of the genomic region comprising haplotype alleles from haplotypes of the haplotype reference panel based on the running sum of the first subset of intermediate allele likelihoods and the running sum of the second subset of intermediate allele likelihoods.

As indicated above, in some cases, determining the sums of intermediate allele likelihoods comprises determining, by a configurable processor and for the marker variant, an initial intermediate allele likelihood from the intermediate allele likelihoods based on an intermediate allele likelihood from the first subset of intermediate allele likelihoods or from the second subset of intermediate allele likelihoods and before summing, for the adjacent marker variant, adjacent-marker intermediate allele likelihoods of the genomic region comprising the haplotype alleles.

Additionally or alternatively, in certain implementations, determining the sums of intermediate allele likelihoods comprises determining, by a configurable processor and for the marker variant, an initial intermediate allele likelihood from the intermediate allele likelihoods based on an intermediate allele likelihood from the first subset of intermediate allele likelihoods or from the second subset of intermediate allele likelihoods and before generating, for the adjacent marker variant, allele likelihoods of the genomic region comprising the haplotype alleles.

As further shown in FIG. 12, the acts 1200 include an act 1210 of generating allele likelihoods based on the sums of intermediate allele likelihoods. In particular, in certain implementations, the act 1210 includes generating allele likelihoods of the genomic region comprising the haplotype alleles based on the sums of intermediate allele likelihoods.

In addition or in the alternative to the acts 1202-1210, in certain implementations, the acts 1000 further include predetermining a first transition-aware allele-likelihood factor corresponding to rows for the first type of haplotype allele and a second transition-aware allele-likelihood factor corresponding to rows for the second type of haplotype allele; and determining a sum of intermediate allele likelihoods based further on the first transition-aware allele-likelihood factor corresponding to the rows for the first type of haplotype allele and the second transition-aware allele-likelihood factor corresponding to rows for the second type of haplotype allele.

Relatedly, in some cases, determining, for the adjacent marker variant, adjacent-marker sums of intermediate allele likelihoods of the genomic region comprising the haplotype alleles; and determining, for the marker variant, the sums of intermediate allele likelihoods based further on a combination of the adjacent-marker sums of intermediate allele likelihoods, the first transition-aware allele-likelihood factor corresponding to the rows for the first type of haplotype allele, and the second transition-aware allele-likelihood factor corresponding to rows for the second type of haplotype allele.

As suggested above, in some cases, the acts 1200 further include multiplying the running sum of the first subset of intermediate allele likelihoods by a first transition-aware allele-likelihood factor; multiplying the running sum of the second subset of intermediate allele likelihoods by a second transition-aware allele-likelihood factor; and determining, for the marker variant, the sums of intermediate allele likelihoods based on the multiplied running sum of the first subset of intermediate allele likelihoods and the multiplied running sum of the second subset of intermediate allele likelihoods.

As further suggested above, in some embodiments, the acts 1200 include predetermining the first transition-aware allele-likelihood factor comprises combining a first allele-likelihood factor for the first type of haplotype allele and a transition linear coefficient for transitioning between haplotypes from the haplotype reference panel; and predetermining the second transition-aware allele-likelihood factor comprises combining a second allele-likelihood factor for the second type of haplotype allele and the transition linear coefficient.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using 7-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the sequencing system 112 or the accelerated genotype-imputation system 106 can include software, hardware, or both. For example, the components of the sequencing system 112 or the accelerated genotype-imputation system 106 can include one or more instructions stored on a computer-readable storage medium and executable by processors of one or more computing devices (e.g., the client device 116). When executed by the one or more processors, the computer-executable instructions of the sequencing system 112 or the accelerated genotype-imputation system 106 can cause the computing devices to perform the bubble detection methods described herein. Alternatively, the components of the sequencing system 112 or the accelerated genotype-imputation system 106 can comprise hardware, such as special purpose processing devices to perform a certain function or group of functions. Additionally, or alternatively, the components of the sequencing system 112 or the accelerated genotype-imputation system 106 can include a combination of computer-executable instructions and hardware.

Furthermore, the components of the accelerated genotype-imputation system 106 performing the functions described herein with respect to the accelerated genotype-imputation system 106 may, for example, be implemented as part of a stand-alone application, as a module of an application, as a plug-in for applications, as a library function or functions that may be called by other applications, and/or as a cloud-computing model. Thus, components of the accelerated genotype-imputation system 106 may be implemented as part of a stand-alone application on a personal computing device or a mobile device. Additionally, or alternatively, the components of the accelerated genotype-imputation system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 13 illustrates a block diagram of a computing device 1300 that may be configured to perform one or more of the processes described above. One will appreciate that one or more computing devices such as the computing device 1300 may implement the accelerated genotype-imputation system 106. As shown by FIG. 13, the computing device 1300 can comprise a processor 1302, a memory 1304, a storage device 1306, an I/O interface 1308, and a communication interface 1310, which may be communicatively coupled by way of a communication infrastructure 1312. In certain embodiments, the computing device 1300 can include fewer or more components than those shown in FIG. 13. The following paragraphs describe components of the computing device 1300 shown in FIG. 13 in additional detail.

In one or more embodiments, the processor 1302 includes hardware for executing instructions, such as those making up a computer program. As an example, and not by way of limitation, to execute instructions for dynamically modifying workflows, the processor 1302 may retrieve (or fetch) the instructions from an internal register, an internal cache, the memory 1304, or the storage device 1306 and decode and execute them. The memory 1304 may be a volatile or non-volatile memory used for storing data, metadata, and programs for execution by the processor(s). The storage device 1306 includes storage, such as a hard disk, flash disk drive, or other digital storage device, for storing data or instructions for performing the methods described herein.

The I/O interface 1308 allows a user to provide input to, receive output from, and otherwise transfer data to and receive data from computing device 1300. The I/O interface 1308 may include a mouse, a keypad or a keyboard, a touch screen, a camera, an optical scanner, network interface, modem, other known I/O devices or a combination of such I/O interfaces. The I/O interface 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1308 is configured to provide graphical data to a display for presentation to a user. The graphical data may be representative of one or more graphical user interfaces and/or any other graphical content as may serve a particular implementation.

The communication interface 1310 can include hardware, software, or both. In any event, the communication interface 1310 can provide one or more interfaces for communication (such as, for example, packet-based communication) between the computing device 1300 and one or more other computing devices or networks. As an example, and not by way of limitation, the communication interface 1310 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1310 may facilitate communications with various types of wired or wireless networks. The communication interface 1310 may also facilitate communications using various communication protocols. The communication infrastructure 1312 may also include hardware, software, or both that couples components of the computing device 1300 to each other. For example, the communication interface 1310 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor;
a memory device; and
a non-transitory computer readable medium comprising instructions that, when executed by the at least one processor, cause the system to:
identify, utilizing a genotype imputation model, a haplotype reference panel for a genomic region of a genomic sample;
access, from the memory device and for a marker variant, a first allele-likelihood factor corresponding to a haplotype allele from the haplotype reference panel and a second allele-likelihood factor corresponding to the haplotype allele;
combine the first allele-likelihood factor and an adjacent-marker intermediate allele likelihood of the genomic region comprising the haplotype allele given an adjacent marker variant to generate an adjacent-marker-factor-aware allele likelihood for the marker variant and a haplotype from the haplotype reference panel;
determine, for the marker variant and the haplotype, an intermediate allele likelihood of the genomic region comprising the haplotype allele based on the adjacent-marker-factor-aware allele likelihood and the second allele-likelihood factor; and
generate, for a set of marker variants corresponding to the genomic region, allele likelihoods of the genomic region comprising haplotype alleles from the haplotype reference panel based on the intermediate allele likelihood.

2. The system of claim 1, further comprising instructions that, when executed by the at least one processor, cause the system to access, from the memory device and for the marker variant, the first allele-likelihood factor and the second allele-likelihood factor by accessing, from the memory device and for the marker variant, a first transition-aware allele-likelihood factor corresponding to the haplotype allele from the haplotype reference panel and a second transition-aware allele-likelihood factor corresponding to the haplotype allele.

3. The system of claim 2, further comprising instructions that, when executed by the at least one processor, cause the system to predetermine the first transition-aware allele-likelihood factor and the second transition-aware allele-likelihood factor before determining one or more intermediate allele likelihoods corresponding to the marker variant as part of a pass across a haplotype matrix.

4. The system of claim 3, further comprising instructions that, when executed by the at least one processor, cause the system to:
predetermine the first transition-aware allele-likelihood factor by combining an allele-likelihood factor for the haplotype allele and a transition constant coefficient for transitioning between haplotypes from the haplotype reference panel; and
predetermine the second transition-aware allele-likelihood factor by combining the allele-likelihood factor and a transition linear coefficient for transitioning between haplotypes from the haplotype reference panel.

5. The system of claim 2, further comprising instructions that, when executed by the at least one processor, cause the system to determine the first transition-aware allele-likelihood factor by combining an allele-likelihood factor and a transition linear coefficient.

6. The system of claim 5, wherein:
the first allele-likelihood factor comprises an allele-likelihood factor for a sample reference haplotype allele or for a sample alternate haplotype allele; and
the second allele-likelihood factor comprises the allele-likelihood factor for the sample reference haplotype allele or for the sample alternate haplotype allele.

7. The system of claim 1, further comprising instructions that, when executed by the at least one processor, cause the system to combine the first allele-likelihood factor and the adjacent-marker intermediate allele likelihood by multiplying a first transition-aware allele-likelihood factor and the adjacent-marker intermediate allele likelihood without further multiplication operations to determine the intermediate allele likelihood.

8. The system of claim 1, further comprising a data flow engine and instructions that, when executed by the at least one processor, cause the system to:
send, from the data flow engine to respective accelerated computation engines of a cluster of accelerated computation engines, respective sets of input values comprising allele-likelihood factors, transition coefficients, and haplotype-allele values; and
determine, by the respective accelerated computation engines and based on the respective sets of input values, respective sets of intermediate allele likelihoods corresponding to respective subsets of marker variants and respective subsets of haplotypes.

9. The system of claim 8, further comprising instructions that, when executed by the at least one processor, cause the system to:
send the respective sets of input values from the data flow engine to the respective accelerated computation engines by:
sending, from the data flow engine to a first accelerated computation engine of the cluster of accelerated computation engines, a first set of input values comprising allele-likelihood factors, transition coefficients, and haplotype-allele values;
sending, from the data flow engine to a second accelerated computation engine of the cluster of accelerated computation engines, a second set of input values comprising allele-likelihood factors, transition coefficients, and haplotype-allele values; and
determine the respective sets of intermediate allele likelihoods by:
determining, by the first accelerated computation engine and based on the first set of input values, a first set of intermediate allele likelihoods corresponding to a first subset of marker variants and a first subset of haplotypes; and
determining, by the second accelerated computation engine and based on the second set of input values, a second set of intermediate allele likelihoods corresponding to a second subset of marker variants and a second subset of haplotypes.

10. A non-transitory computer-readable medium storing instructions that, when executed by at least one processor, cause a computing device to:
identify, utilizing a genotype imputation model, a haplotype reference panel for a genomic region of a genomic sample;
access, from a memory device and for a marker variant, a first transition-aware allele-likelihood factor corresponding to a haplotype allele from the haplotype reference panel and a second transition-aware allele-likelihood factor corresponding to the haplotype allele;
combine, by a configurable processor, the first transition-aware allele-likelihood factor and an adjacent-marker intermediate allele likelihood of the genomic region comprising the haplotype allele given an adjacent marker variant to generate an adjacent-marker-transition-factor-aware allele likelihood for the marker variant and a haplotype from the haplotype reference panel;
determine, by the configurable processor and for the marker variant and the haplotype, an intermediate allele likelihood of the genomic region comprising the haplotype allele based on the adjacent-marker-transition-factor-aware allele likelihood and the second transition-aware allele-likelihood factor; and
generate, by the configurable processor and for a set of marker variants corresponding to the genomic region, allele likelihoods of the genomic region comprising haplotype alleles from the haplotype reference panel based on the intermediate allele likelihood.

11. The non-transitory computer-readable medium of claim 10, further comprising instructions that, when executed by the at least one processor, cause the computing device to predetermine the first transition-aware allele-likelihood factor and the second transition-aware allele-likelihood factor before determining one or more intermediate allele likelihoods corresponding to the marker variant.

12. The non-transitory computer-readable medium of claim 11, further comprising instructions that, when executed by the at least one processor, cause the computing device to:
predetermine the first transition-aware allele-likelihood factor by combining an allele-likelihood factor for the haplotype allele and a transition constant coefficient for transitioning between haplotypes from the haplotype reference panel; and
predetermine the second transition-aware allele-likelihood factor by combining the allele-likelihood factor and a transition linear coefficient for transitioning between haplotypes from the haplotype reference panel.

13. The non-transitory computer-readable medium of claim 10, further comprising instructions that, when executed by the at least one processor, cause the computing device to combine the first transition-aware allele-likelihood factor and the adjacent-marker intermediate allele likelihood by multiplying the first transition-aware allele-likelihood factor and the adjacent-marker intermediate allele likelihood without further multiplication operations to determine the intermediate allele likelihood.

14. The non-transitory computer-readable medium of claim 10, further comprising instructions that, when executed by the at least one processor, cause the computing device to:
access the second transition-aware allele-likelihood factor as part of a summed-adjacent-marker transition-aware allele-likelihood factor; and
determine the intermediate allele likelihood based on the adjacent-marker-transition-factor-aware allele likelihood and the summed-adjacent-marker transition-aware allele-likelihood factor.

15. The non-transitory computer-readable medium of claim 14, further comprising instructions that, when executed by the at least one processor, cause the computing device to predetermine the summed-adjacent-marker transition-aware allele-likelihood factor by combining an allele-likelihood factor for the haplotype allele, a transition constant coefficient for transitioning between haplotypes from the haplotype reference panel, and summed adjacent-marker intermediate allele likelihoods for the adjacent marker variant.

16. The non-transitory computer-readable medium of claim 15, wherein the allele-likelihood factor for the haplotype allele comprises a reference allele-likelihood factor for a sample reference haplotype allele or an alternate allele-likelihood factor for a sample alternate haplotype allele.

17. A method comprising:
identifying, utilizing a genotype imputation model, a haplotype reference panel for a genomic region of a genomic sample;
accessing, from a memory device and for a marker variant, a first transition-aware allele-likelihood factor corresponding to a haplotype allele from the haplotype reference panel and a second transition-aware allele-likelihood factor corresponding to the haplotype allele;
combining, by a configurable processor, the first transition-aware allele-likelihood factor and an adjacent-marker intermediate allele likelihood of the genomic region comprising the haplotype allele given an adjacent marker variant to generate an adjacent-marker-transition-factor-aware allele likelihood for the marker variant and a haplotype from the haplotype reference panel;
determining, by the configurable processor and for the marker variant and the haplotype, an intermediate allele likelihood of the genomic region comprising the haplotype allele based on the adjacent-marker-transition-factor-aware allele likelihood and the second transition-aware allele-likelihood factor; and
generating, by the configurable processor and for a set of marker variants corresponding to the genomic region, allele likelihoods of the genomic region comprising haplotype alleles from the haplotype reference panel based on the intermediate allele likelihood.

18. The method of claim 17, wherein the genotype imputation model comprises a hidden Markov genotype imputation model.

19. The method of claim 17, wherein the configurable processor comprises an application-specific integrated circuit (ASIC), an application-specific standard product (ASSP), a coarse-grained reconfigurable array (CGRA), or a field programmable gate array (FPGA).

20. The method of claim 17, wherein the memory device comprises dynamic random-access memory (DRAM), dynamic random-access memory (SRAM), or a cache memory device.
